(19) United States
(12) Patent Application Publication
(10) Pub. No.: US 2012/0115877 A1
(43) Pub. Date: May 10, 2012

(54) Facially Amphiphilic Compounds, Compositions, And Uses Thereof In Treating Cancer

(75) Inventors: Richard W. Scott, Radnor, PA (US); Dylan Clements, Radnor, PA (US); Yongjiang Xu, Radnor, PA (US); Haizhong Tang, Radnor, PA (US); Carol Mulrooney, Radnor, PA (US); Ehab Khalil, Radnor, PA (US); Damian Weaver, Radnor, PA (US); Xiaodong Fan, Radnor, PA (US)

(73) Assignee: POLYMEDIX, INC., Radnor, PA (US)

(21) Appl. No.: 13/214,177

(22) Filed: Aug. 20, 2011

Related U.S. Application Data
(60) Provisional application No. 61/375,804, filed on Aug. 21, 2010.

(51) Int. Cl. A61K 31/496; A61K 31/4025; A61P 35/00; A61K 31/343; A61P 35/02; A61K 31/505; A61K 31/381

(52) U.S. Cl. 514/252.11; 514/256; 514/422; 514/443; 514/468

(57) ABSTRACT

The present invention discloses compositions of facially amphiphilic compounds and their use in methods for treating or reducing cancers in animals, such as humans.

### FIELD OF THE INVENTION

The present invention relates to compositions of facially amphiphilic compounds and their use in methods for treating cancers in animals, such as humans.

### BACKGROUND

Antimicrobial peptides represent a large and growing class of biologically interesting compounds. They represent the first line of defense against microbes for many species including plants, insects, worms and mammals. In mammals, the peptides are produced and secreted in skin, musosal surfaces and neutrophils. There are many different classes of natural host defense peptides but, in general, most contain between 20-40 amino acid residues and adopt a facially amphiphilic secondary structure with positively charged groups segregated to one side of the secondary structure and hydrophobic groups on the opposite surface. These structures can be described as facially amphiphilic regardless of whether the secondary structure is a helix or sheet type fold. It is the overall physiochemical properties that are responsible for biological activity of these peptides and not the precise amino acid sequence.

The specificity of the cytotoxic activity of the cationic and amphiphilic peptides for bacteria over mammalian cells is most likely related to fundamental differences between the two membrane types: bacteria have a large proportion of negatively charges phospholipids headgroups on their surface, while the outer leaflet of animal cells is composed mainly of neutral lipids. Also, the presence of cholesterol in the animal cell membrane appears to reduce the activity of the antimicrobial peptides. Several mechanisms have been proposed for the process of cell killing. In the carpet mechanism, peptides aggregate parallel to the membrane surface, leading to thinning and ultimately rupture of the membrane. The so-called barrel-stave mechanism suggests that the bound peptides on the cell surface self-associate into transmembrane helical bundles that form stable aqueous pores in the membrane. A third explanation is that the peptides initially bind only to the outer leaflet of the bilayer that leads to an increase in the lateral surface pressure of the outer leaflet relative to the inner leaflet of the bilayer. This pressure imbalance results in translocation of the peptides into the interior of the bilayer with concomitant formation of transient openings in the membrane. Formation of these transient pores would allow hydration of the polar sidechains of the peptide and leakage of cellular contents. Most antimicrobial peptides probably act by more than one of these mechanisms.

It has been found that several of the antimicrobial peptides, including the magainins and human cathelicidin LL-37, are more toxic to tumor cells than normal cells. See Baker et al., Cancer Res., 1993, 53, 3052-3057; Cruciani et al., Proc. Natl. Acad. Sci. USA, 1991, 88, 3792-3796; and Okumura et al., Cancer Lett., 2004, 212, 185-194.

This preferential cytotoxic activity has been attributed to a slightly higher content of negatively charged phosphatidyl serine in the tumor cell membrane resulting in tumor cells having a slightly higher negative charge on their surface in comparison to normal animal cells. Tumor cells have other differences that may also be involved in the selectivity of the cationic amphiphilic peptides, including a higher content of O-glycosylated mucines in their cell membranes and a higher intracellular negative potential (Papo et al., Biochemistry, 2003, 42, 9346-9354).

Several synthetic peptides and peptoids have been synthesized to mimic the activity of the natural host defense proteins (DeGrado, Adv. Protein Chem., 1988, 51-124; Hamuro et al., J. Am. Chem. Soc., 1999, 121, 12200-12201; Porter et al., Nature (London), 2000, 404, 565; Porter et al., J. Am. Chem. Soc., 2002, 124, 7324-7330; Liu et al., J. Am. Chem. Soc., 2001, 123, 7553-7559; Patch et al., J. Am. Chem. Soc., 2003, 125, 12092-12093; and Seurynck et al., Biophysical Journal, 2003, 84, 298A-298A) and several of these these have been shown to selectively kill tumorigenic cells (Papo et al., Biochemistry, 2003, 42, 9346-9354; Papo et al., Cancer Res., 2004, 64, 5779-5786; and Shin et al., Biochim. Biophys. Acta, 2000, 1463, 209-218).

A series of nonpeptidic mimics of the natural antimicrobial peptides have been developed that are polymers, oligomers and small molecules comprised of non-natural building blocks. See, Tew et al., Proc. Natl. Acad. Sci. U.S.A., 2002, 99, 5110-5116; Arnt et al., J. Polym. Sci., 2004, Part A 42, 3860-3864; and Liu et al., Angew Chem Int Ed Engl., 2004, 43, 1158-1162. See also, WIPO Publ. No. WO 2004/082634; WIPO Publ. No. 02/100295, and WIPO Publ. No. 02/072007. Many of these compounds are significantly smaller and easier to prepare than the natural antimicrobial peptides and peptidic mimetics. The shortest of these oligomers have molecular weights typical of small molecule drugs. They have the same mechanism of action as magainin, are highly potent and have a broad spectrum of activity, killing gram-positive, gram-negative and antibiotic-resistant human pathogens. Relative to the antimicrobial peptides, the non-peptidic mimetics are significantly less toxic towards human erythrocytes, much less expensive to prepare, and more stable. Furthermore, recent results in an animal model of bacterial infection have demonstrated robust in vivo efficacy for an initial set of compounds, demonstrating the ability of the compounds to access an infected tissue when administered in the bloodstream.

### SUMMARY OF THE INVENTION

The present invention provides compositions of facially amphiphilic compounds and methods for their use in treating cancers in animals, such as humans.

The present invention is also directed to methods of treating cancer in an animal in need thereof comprising administering to the animal an effective amount of a pharmaceutical composition comprising a compound of the invention, or an acceptable salt or solvate thereof, and a pharmaceutically acceptable carrier or diluent.

The present invention is also directed to methods of killing or inhibiting the growth of a cancer cell comprising contacting the cancer cell with an effective amount of a compound of the invention, or an acceptable salt or solvate thereof.

The present invention is further directed to methods of reducing cancer in an animal comprising administering to the animal an effective amount of a compound of the invention, or an acceptable salt or solvate thereof.

The present invention is also directed to methods of inhibiting tumor growth comprising contacting the tumor with an effective amount of a compound of the invention, or a acceptable salt or solvate thereof.

The present invention is also directed to methods of treating or preventing the spread or metastasis of cancer in an animal comprising administering to the animal an effective amount of a compound of the invention, or an acceptable salt or solvate thereof.

The present invention is further directed to methods of treating an animal afflicted with a tumor or cancer comprising administering to the animal an effective amount of a compound of the invention, or an acceptable salt or solvate thereof.

In particular, the present invention provides, inter alia, the following embodiments:

a) A method for treating cancer in an animal in need thereof comprising administering to the animal an effective amount of a compound or pharmaceutically acceptable salt thereof; wherein the compound or pharmaceutically acceptable salt administered is a compound of Formula I:

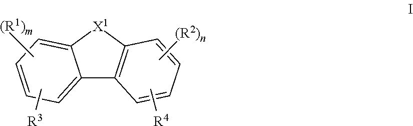

or pharmaceutically acceptable salt thereof, wherein: X¹ is O, S, S(═O), or S(═O)₂; R¹ and R² are, independently, halo, C₁₋₄ alkyl, C₁₋₄ alkoxy, CN, C₁₋₄ haloalkyl, or C₁₋₄ haloalkoxy; R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶; each R⁵ is independently —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, —(CH₂)ₜ₇—NH₂, or NR⁷R⁸; each R⁶ is independently halo, OH, C₁₋₄ alkyl, C₁₋₄ alkoxy, CN, C₁₋₄ haloalkyl, or C₁₋₄ haloalkoxy; R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl; m is 0 or 1; n is 0 or 1; each t1 is independently 2 or 3; each t2 is independently 1, 2, or 3; each t3 is independently 2 or 3; each t4 is independently 2 or 3; each t5 is independently 2 or 3; each t6 is independently 2 or 3; and each t7 is independently 2 or 3; or the compound or pharmaceutically acceptable salt administered is a compound of Formula II:

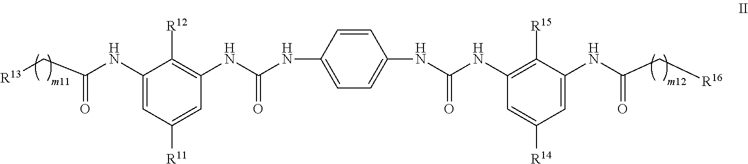

or pharmaceutically acceptable salt thereof, wherein: R¹¹ and R¹⁴ are, independently, H, Cl, CN, methyl, CH₂F, CHF₂, or CF₃; R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂; R¹³ and R¹⁶ are, independently, —NH₂, —NH—(CH₂)ₘ₁₃—NH₂, —NHC(═NH)NH₂, or —NH—(CH₂)ₘ₁₄—NHC(═NH)NH₂; each R¹⁷ is independently pyrrolidinyl, piperidinyl, morpholinyl, or piperazinyl, each optionally substituted with 1 or 2 C₁₋₄ alkyl; each of t11, t12, t13, and t14 is independently 2 or 3; each of m11 and m12 is independently 3, 4, or 5; each of m13 and m14 is independently 1, 2, 3, 4, or 5; or the compound or pharmaceutically acceptable salt administered is a compound of Formula III:

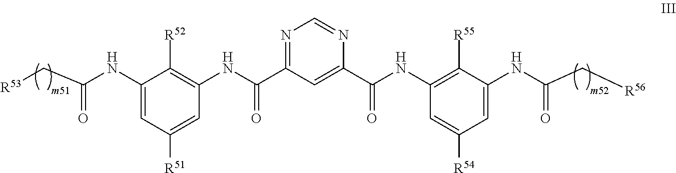

or pharmaceutically acceptable salt thereof, wherein: R⁵¹ and R⁵⁴ are, independently, H, Cl, CN, methyl, CH₂F, CHF₂, or CF₃; R⁵² and R⁵⁵ are, independently, —S—(CH₂)ₜ₅₁—NH₂ or —(CH₂)ₜ₅₂—NH₂; R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂; each of t51 and t52 is independently 2 or 3; each of m51 and m52 is independently 3, 4, or 5; and each of m53 and m54 is independently 1, 2, 3, 4, or 5; or the compound or pharmaceutically acceptable salt administered is a compound of Formula IV or IVa:

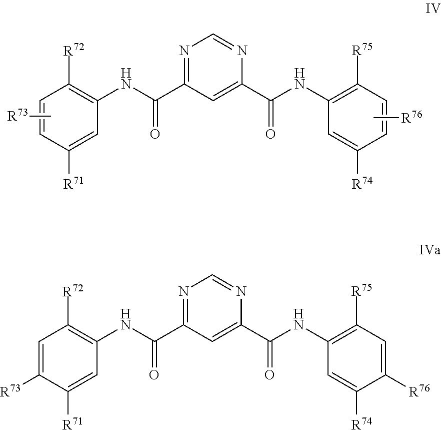

or pharmaceutically acceptable salt thereof, wherein: R⁷¹ and R⁷⁴ are, independently, H, Cl, CN, methyl, CH₂F, CHF₂, or CF₃; R⁷² and R⁷⁵ are, independently, —S—(CH₂)ₜ₇₁—NH₂, —(CH₂)ₜ₇₂—NH₂, or —O—(CH₂)ₜ₇₃—NH₂; R⁷³ and R⁷⁶ are, independently, —S—(CH₂)ₜ₇₄—NH₂, —(CH₂)ₜ₇₅—NH₂, or —O—(CH₂)ₜ₇₆—NH₂; t71 and t74 are independently 2 or 3; t72 and t75 are independently 2 or 3; and t73 and t76 are independently 2 or 3.

In some embodiments, the compound or pharmaceutically acceptable salt thereof administered is a compound of Formula I or pharmaceutically acceptable salt thereof. In some embodiments, R¹ and R² are, independently, halo, methyl, or C₁ haloalkyl. In some embodiments, R¹ and R² are, independently, Cl, Br, methyl, CH₂F, CHF₂, or CF₃. In some embodiments, m is 0. In some embodiments, m is 1. In some embodiments, n is 0. In some embodiments, n is 1. In some embodiments, R³ and R⁴ are, independently, phenyl or pyridinyl, each substituted with R⁵ and optionally substituted with R⁶. In some embodiments, R³ and R⁴ are, independently, phenyl substituted with R⁵ and optionally substituted with R⁶. In some embodiments, R³ and R⁴ are, independently, pyridinyl substituted with R⁵ and optionally substituted with R⁶. In some embodiments, each R⁵ is independently —(CH₂)₂—NH₂, —(CH₂)₃—NH₂, —S—(CH₂)₂—NH₂, —O—(CH₂)₃—NH₂, or piperazin-1-yl.

In some embodiments, the compound of Formula I or pharmaceutically acceptable salt thereof is a compound Formula Ia:

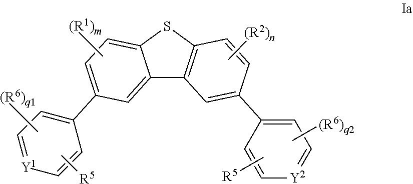

or pharmaceutically acceptable salt thereof, wherein: R¹ and R² are, independently, Cl, Br, methyl, CH₂F, CHF₂, or CF₃; each R⁵ is independently —(CH₂)₂—NH₂, —(CH₂)₃—NH₂, —S—(CH₂)₂—NH₂, —O—(CH₂)₃—NH₂, or piperazin-1-yl; each R⁶ is independently, Cl, Br, methyl, CH₂F, CHF₂, or CF₃; Y¹ is N or CH; Y² is N or CH; m is 0 or 1; n is 0 or 1; q1 is 0 or 1; and q2 is 0 or 1. In some embodiments, R¹ and R² are, independently, Cl, Br, methyl, or CF₃. In some embodiments, R¹ and R² are, independently, Cl or Br. In some embodiments, m is 0. In some embodiments, n is 0. In some embodiments, each R⁵ is independently —(CH₂)₂—NH₂, —(CH₂)₃—NH₂, or piperazin-1-yl. In some embodiments, each R⁵ is independently —(CH₂)₃—NH₂ or piperazin-1-yl. In some embodiments, q1 is 0. In some embodiments, q2 is 0.

In some embodiments, the compound of Formula Ia or pharmaceutically acceptable salt thereof is a compound Formula Ia-1:

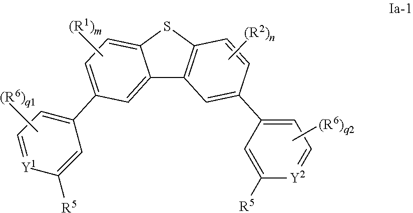

or pharmaceutically acceptable salt thereof. In some embodiments, the compound of Formula Ia-1 or pharmaceutically acceptable salt thereof is a compound of Formula Ia-1-1:

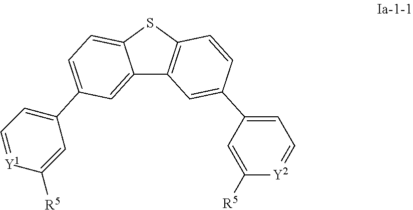

or pharmaceutically acceptable salt thereof. In some embodiments, the compound of Formula Ia or pharmaceutically acceptable salt thereof is a compound selected from:

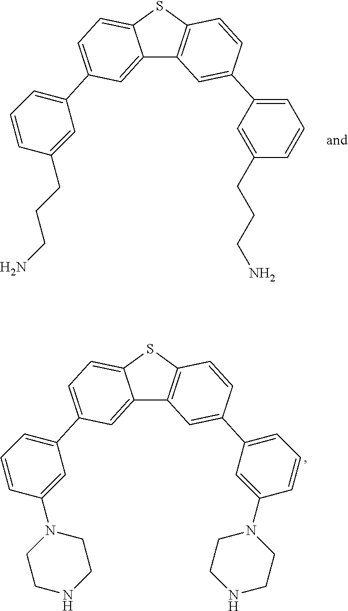

or pharmaceutically acceptable salt thereof.

In some embodiments, the compound of Formula I or pharmaceutically acceptable salt thereof is a compound Formula Ib:

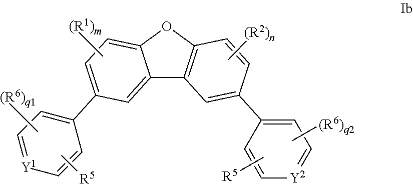

or pharmaceutically acceptable salt thereof, wherein: R¹ and R² are, independently, Cl, Br, methyl, CH₂F, CHF₂, or CF₃; each R⁵ is independently —(CH₂)₂—NH₂, —(CH₂)₃—NH₂, —S—(CH₂)₂—NH₂, —O—(CH₂)₃—NH₂, or piperazin-1-yl; each R⁶ is independently, Cl, Br, methyl, CH₂F, CHF₂, or CF₃; Y¹ is N or CH; Y² is N or CH; m is 0 or 1; n is 0 or 1; q1 is 0 or 1; and q2 is 0 or 1. In some embodiments, R¹ and R² are, independently, Cl, Br, methyl, or CF₃. In some embodiments, R¹ and R² are, independently, Cl or Br. In some embodiments, m is 0. In some embodiments, n is 0. In some embodiments, each R⁵ is independently —(CH₂)₂—NH₂, —(CH₂)₃—NH₂, or piperazin-1-yl. In some embodiments, each R⁵ is independently —(CH₂)₃—NH₂ or piperazin-1-yl. In some embodiments, q1 is 0. In some embodiments, q2 is 0.

In some embodiments, the compound of Formula Ib or pharmaceutically acceptable salt thereof is a compound Formula Ib-1:

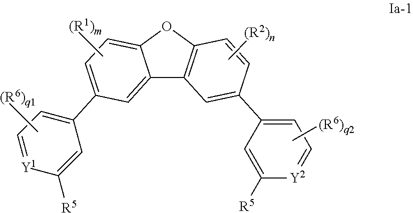

or pharmaceutically acceptable salt thereof. In some embodiments, the compound of Formula Ib-1 or pharmaceutically acceptable salt thereof is a compound of Formula Ib-1-1:

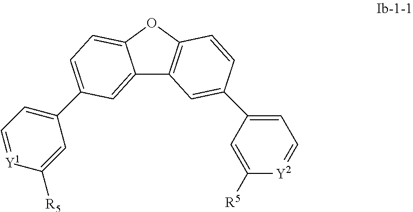

or pharmaceutically acceptable salt thereof. In some embodiments, the compound of Formula Ib or pharmaceutically acceptable salt thereof is a compound that is

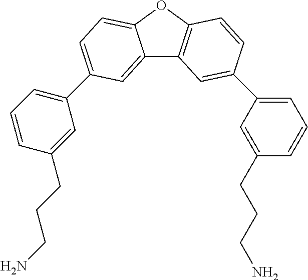

or pharmaceutically acceptable salt thereof.

In some embodiments, the compound or pharmaceutically acceptable salt thereof administered is a compound of Formula II or pharmaceutically acceptable salt thereof. In some embodiments, R¹¹ and R¹⁴ are, independently, methyl, CH₂F, CHF₂, or CF₃. In some embodiments, R¹¹ and R¹⁴ are, independently, methyl or CF₃. In some embodiments, R¹¹ and R¹⁴ are each CF₃. In some embodiments, R¹² and R¹⁵ are, independently, —S—(CH₂)₂—NH₂, —O—(CH₂)₂—NH₂, —O—(CH₂)₃—NH₂, —(CH₂)₃—NH₂, or —O—R¹⁷; and each R¹⁷ is independently pyrrolidin-2-yl, pyrrolidin-3-yl, piperidin-2-yl, piperidin-3-yl, piperidin-4-yl, or piperazin-2-yl. In some embodiments, R¹² and R¹⁵ are, independently, —S—(CH₂)₂—NH₂, —O—(CH₂)₃—NH₂, —(CH₂)₃—NH₂, or —O—R¹⁷; and each R¹⁷ is independently pyrrolidin-3-yl or piperidin-3-yl. In some embodiments, R¹² and R¹⁵ are, each —O—R¹⁷; and each R¹⁷ is pyrrolidin-3-yl. In some embodiments, each of m11 and m12 is 4. In some embodiments, R¹³ and R¹⁶ are, independently, —NHC(═NH)NH₂ or —NH—(CH₂)ₘ₁₄—NHC(═NH)NH₂; and each m14 is 1, 2, or 3. In some embodiments, R¹³ and R¹¹⁶ are each —NHC(═NH)NH₂. In some embodiments, the compound of Formula II or pharmaceutically acceptable salt thereof is a compound that is:

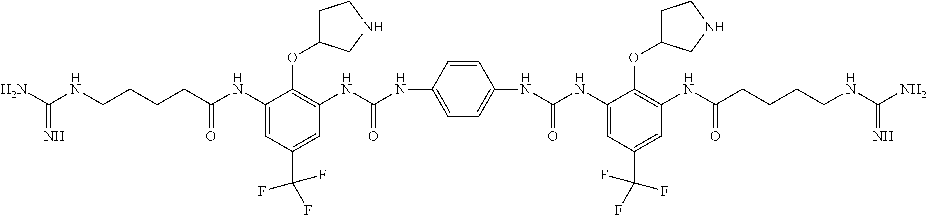

or pharmaceutically acceptable salt thereof.

In some embodiments, the compound or pharmaceutically acceptable salt thereof administered is a compound of Formula III or pharmaceutically acceptable salt thereof. In some embodiments, R⁵¹ and R⁵⁴ are, independently, methyl, CH₂F, CHF₂, or CF₃. In some embodiments, R⁵¹ and R⁵⁴ are, independently, methyl or CF₃. In some embodiments, R⁵¹ and R⁵⁴ are each CF₃. In some embodiments, R⁵² and R⁵⁵ are, independently, —S—(CH₂)₂—NH₂ or —(CH₂)₃—NH₂. In some embodiments, R⁵² and R⁵⁵ are each —S—(CH₂)₂—NH₂. In some embodiments, each of m51 and m52 is 4. In some embodiments, R⁵³ and R⁵⁶ are, independently, —NHC(═NH)NH₂ or —NH—(CH₂)ₘ₅₄—NHC(═NH)NH₂; and each m54 is 1, 2, or 3. In some embodiments, R⁵³ and R⁵⁶ are each —NHC(═NH)NH₂. In some embodiments, the compound of Formula III or pharmaceutically acceptable salt thereof is a compound that is:

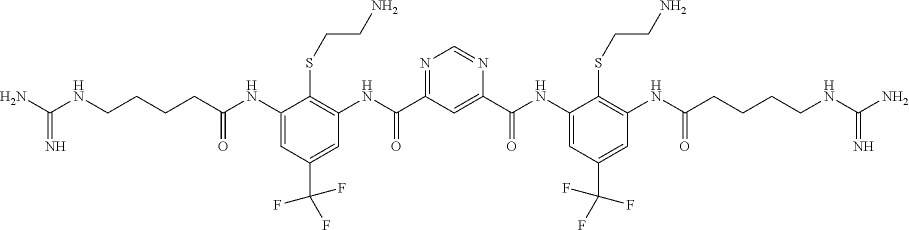

or pharmaceutically acceptable salt thereof.

In some embodiments, the compound or pharmaceutically acceptable salt thereof administered is a compound of Formula IV or pharmaceutically acceptable salt thereof. In some embodiments, the compound or pharmaceutically acceptable salt thereof administered is a compound of Formula IVa or pharmaceutically acceptable salt thereof. In some embodiments, R⁷¹ and R⁷⁴ are, independently, methyl, CH₂F, CHF₂, or CF₃. In some embodiments, R⁷¹ and R⁷⁴ are, independently, methyl or CF₃. In some embodiments, R⁷¹ and R⁷⁴ are each CF₃. In some embodiments, R⁷² and R⁷⁵ are, independently, —S—(CH₂)₂—NH₂, —S(CH₂)₃—NH₂, —(CH₂)₃—NH₂, or —O(CH₂)₃—NH₂. In some embodiments, R⁷² and R⁷⁵ are, independently, —S—(CH₂)₂—NH₂, —(CH₂)₃—NH₂, or —O(CH₂)₃—NH₂. In some embodiments, R⁷² and R⁷⁵ are each —S—(CH₂)₂—NH₂. In some embodiments, R⁷³ and R⁷⁶ are, independently, —S—(CH₂)₂—NH₂, —S(CH₂)₃—NH₂, —(CH₂)₃—NH₂, or —O(CH₂)₃—NH₂. In some embodiments, R⁷³ and R⁷⁶ are, independently, —S—(CH₂)₂—NH₂, —(CH₂)₃—NH₂, or —O(CH₂)₃—NH₂. In some embodiments, R⁷³ and R⁷⁶ are each —S—(CH₂)₂—NH₂. In some embodiments, the compound of Formula IV or IVa, or pharmaceutically acceptable salt thereof, is a compound that is:

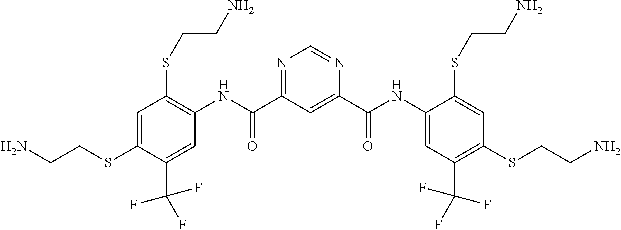

or pharmaceutically acceptable salt thereof.

In some embodiments, the cancer is selected from leukemia, melanoma, lung cancer, colon cancer, brain cancer, ovary cancer, breast cancer, prostate cancer, and kidney cancer.

b) A method for killing or inhibiting growth of a cancer cell comprising contacting the cancer cell with an effective amount of a compound or pharmaceutically acceptable salt thereof; wherein the compound or pharmaceutically acceptable salt is a compound of Formula I:

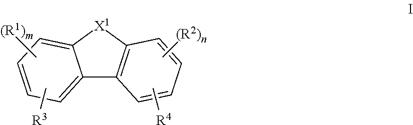

or pharmaceutically acceptable salt thereof, wherein: X¹ is O, S, S(═O), or S(═O)₂; R¹ and R² are, independently, halo, C₁₋₄ alkyl, C₁₋₄ alkoxy, CN, C₁₋₄ haloalkyl, or C₁₋₄ haloalkoxy; R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶; each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸; each R⁶ is independently, halo, OH, C₁₋₄ alkyl, C₁₋₄ alkoxy, CN, C₁₋₄ haloalkyl, or C₁₋₄ haloalkoxy; R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl; m is 0 or 1; n is 0 or 1; each t1 is independently 2 or 3; each t2 is independently 1, 2, or 3; each t3 is independently 2 or 3; each t4 is independently 2 or 3; and each t5 is independently 2 or 3; or the compound or pharmaceutically acceptable salt is a compound of Formula II:

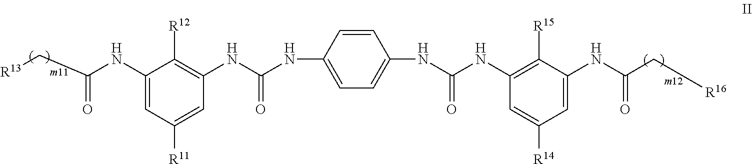

or pharmaceutically acceptable salt thereof, wherein: R¹¹ and R¹⁴ are, independently, H, Cl, CN, methyl, CH₂F, CHF₂, or CF₃; R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂; R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂; each R¹⁷ is independently, pyrrolidinyl, piperidinyl, morpholinyl, or piperazinyl, each optionally substituted with 1 or 2 C₁₋₄ alkyl; each of t11, t12, t13, and t14 is independently 2 or 3; each of m11 and m12 is independently 3, 4, or 5; each of m13 and m14 is independently 1, 2, 3, 4, or 5; or the compound or pharmaceutically acceptable salt is a compound of Formula III:

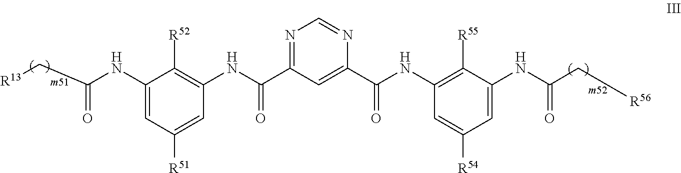

or pharmaceutically acceptable salt thereof, wherein: R⁵¹ and R⁵⁴ are, independently, H, Cl, CN, methyl, CH₂F, CHF₂, or CF₃; R⁵² and R⁵⁵ are, independently, —S—(CH₂)ₜ₅₁—NH₂ or —(CH₂)ₜ₅₂—NH₂; R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂; each of t51 and t52 is independently 2 or 3; each of m51 and m52 is independently 3, 4, or 5; and each of m53 and m54 is independently 1, 2, 3, 4, or 5; or the compound or pharmaceutically acceptable salt is a compound of Formula IV or IVa:

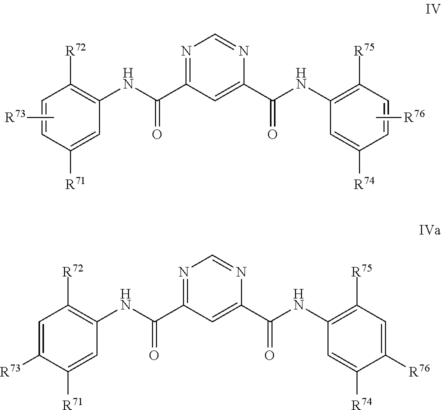

or pharmaceutically acceptable salt thereof, wherein: R⁷¹ and R⁷⁴ are, independently, H, Cl, CN, methyl, CH₂F, CHF₂, or CF₃; R⁷² and R⁷⁵ are, independently, —S—(CH₂)ₜ₇₁—NH₂, —(CH₂)ₜ₇₂—NH₂, or —O—(CH₂)ₜ₇₃—NH₂; R⁷³ and R⁷⁶ are, independently, —S—(CH₂)ₜ₇₄—NH₂, —(CH₂)ₜ₇₅—NH₂, or —O—(CH₂)ₜ₇₆—NH₂; t71 and t74 are independently 2 or 3; t72 and t75 are independently 2 or 3; and t73 and t76 are independently 2 or 3.

c) A method for reducing cancer in an animal comprising administering to said animal an effective amount of a compound or pharmaceutically acceptable salt thereof; wherein the compound or pharmaceutically acceptable salt administered is a compound of Formula I:

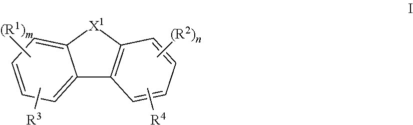

or pharmaceutically acceptable salt thereof, wherein: X¹ is O, S, S(═O), or S(═O)₂; R¹ and R² are, independently, halo, C₁₋₄ alkyl, C₁₋₄ alkoxy, CN, C₁₋₄ haloalkyl, or C₁₋₄ haloalkoxy; R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶; each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸; each R⁶ is independently, halo, OH, C₁₋₄ alkyl, C₁₋₄ alkoxy, CN, C₁₋₄ haloalkyl, or C₁₋₄ haloalkoxy; R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl; m is 0 or 1; n is 0 or 1; each t1 is independently 2 or 3; each t2 is independently 1, 2, or 3; each t3 is independently 2 or 3; each t4 is independently 2 or 3; and each t5 is independently 2 or 3; or the compound or pharmaceutically acceptable salt administered is a compound of Formula II:

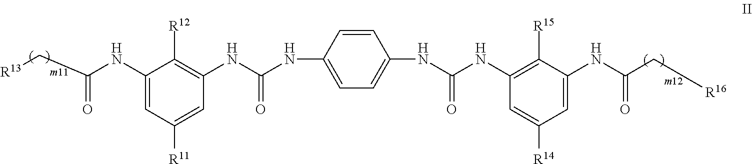

or pharmaceutically acceptable salt thereof, wherein: R¹¹ and R¹⁴ are, independently, H, Cl, CN, methyl, CH₂F, CHF₂, or CF₃; R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂; R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂; each R¹⁷ is independently, pyrrolidinyl, piperidinyl, morpholinyl, or piperazinyl, each optionally substituted with 1 or 2 C₁₋₄ alkyl; each of t11, t12, t13, and t14 is independently 2 or 3; each of m11 and m12 is independently 3, 4, or 5; each of m13 and m14 is independently 1, 2, 3, 4, or 5; or the compound or pharmaceutically acceptable salt administered is a compound of Formula III:

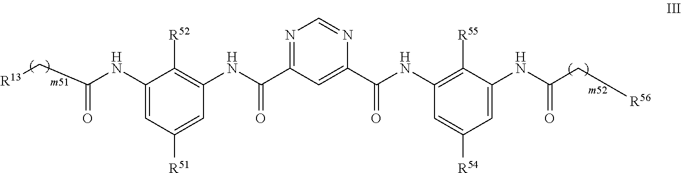

or pharmaceutically acceptable salt thereof, wherein: R⁵¹ and R⁵⁴ are, independently, H, Cl, CN, methyl, CH₂F, CHF₂, or CF₃; R⁵² and R⁵⁵ are, independently, —S—(CH₂)ₜ₅₁—NH₂ or —(CH₂)ₜ₅₂—NH₂; R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂; each of t51 and t52 is independently 2 or 3; each of m51 and m52 is independently 3, 4, or 5; and each of m53 and m54 is independently 1, 2, 3, 4, or 5; or the compound or pharmaceutically acceptable salt administered is a compound of Formula IV or IVa:

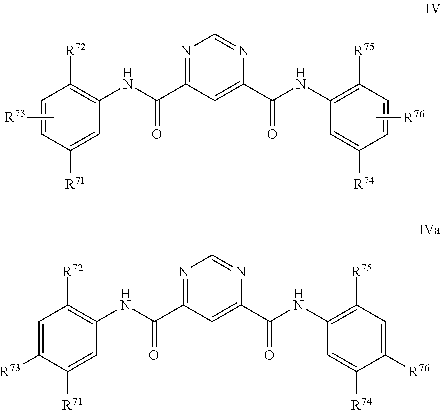

or pharmaceutically acceptable salt thereof, wherein: R⁷¹ and R⁷⁴ are, independently, H, Cl, CN, methyl, CH₂F, CHF₂, or CF₃; R⁷² and R⁷⁵ are, independently, —S—(CH₂)ₜ₇₁′—NH₂, —(CH₂)ₜ₇₂—NH₂, or —O—(CH₂)ₜ₇₃—NH₂; R⁷³ and R⁷⁶ are, independently, —S—(CH₂)ₜ₇₄—NH₂, —(CH₂)ₜ₇₅—NH₂, or —O—(CH₂)ₜ₇₆—NH₂; t71 and t74 are independently 2 or 3; t72 and t75 are independently 2 or 3; and t73 and t76 are independently 2 or 3.

d) A method of reducing cancer in an animal comprising administering to said animal an effective amount of a compound or pharmaceutically acceptable salt thereof; wherein the compound or pharmaceutically acceptable salt administered is a compound of Formula I:

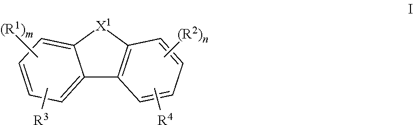

or pharmaceutically acceptable salt thereof, wherein: X¹ is O, S, S(═O), or S(═O)₂; R¹ and R² are, independently, halo, C₁₋₄ alkyl, C₁₋₄ alkoxy, CN, C₁₋₄ haloalkyl, or C₁₋₄ haloalkoxy; R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶; each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂₅—S—(CH₂)ₜ₅—NH₂, —O—(CH₂)ₜ₆—NH₂, or NR⁷R⁸; each R⁶ is independently, halo, OH, C₁₋₄ alkyl, C₁₋₄ alkoxy, CN, C₁₋₄ haloalkyl, or C₁₋₄ haloalkoxy; R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl; m is 0 or 1; n is 0 or 1; each t1 is independently 2 or 3; each t2 is independently 1, 2, or 3; each t3 is independently 2 or 3; each t4 is independently 2 or 3; and each t5 is independently 2 or 3; or the compound or pharmaceutically acceptable salt administered is a compound of Formula II:

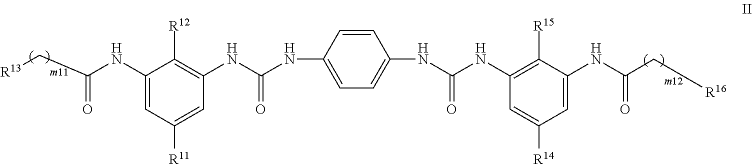

or pharmaceutically acceptable salt thereof, wherein: R¹¹ and R¹⁴ are, independently, H, Cl, CN, methyl, CH₂F, CHF₂, or CF₃; R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂; R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂; each R¹⁷ is independently, pyrrolidinyl, piperidinyl, morpholinyl, or piperazinyl, each optionally substituted with 1 or 2 C₁₋₄ alkyl; each of t11, t12, t13, and t14 is independently 2 or 3; each of m11 and m12 is independently 3, 4, or 5; each of m13 and m14 is independently 1, 2, 3, 4, or 5; or the compound or pharmaceutically acceptable salt administered is a compound of Formula III:

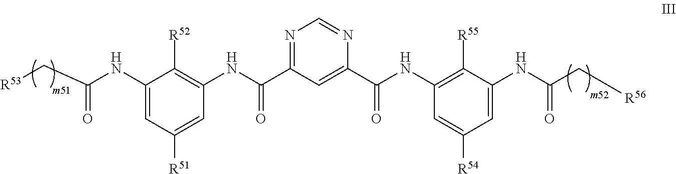

or pharmaceutically acceptable salt thereof, wherein: R⁵¹ and R⁵⁴ are, independently, H, Cl, CN, methyl, CH₂F, CHF₂, or CF₃; R⁵² and R⁵⁵ are, independently, —S—(CH₂)ₜ₅₁—NH₂ or —(CH₂)ₜ₅₂—NH₂; R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂; each of t51 and t52 is independently 2 or 3; each of m51 and m52 is independently 3, 4, or 5; and each of m53 and m54 is independently 1, 2, 3, 4, or 5; or the compound or pharmaceutically acceptable salt administered is a compound of Formula IV or IVa:

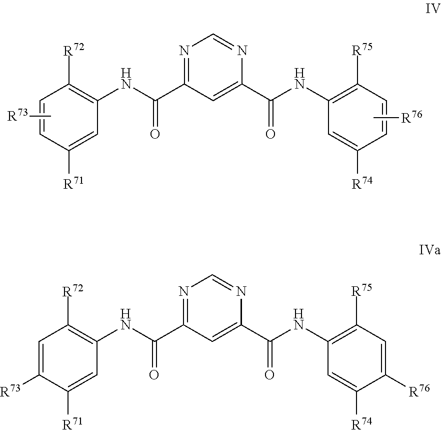

or pharmaceutically acceptable salt thereof, wherein: R⁷¹ and R⁷⁴ are, independently, H, Cl, CN, methyl, CH₂F, CHF₂, or CF₃; R⁷² and R⁷⁵ are, independently, —S—(CH₂)ₜ₇₁′—NH₂, —(CH₂)ₜ₇₂—NH₂, or —O—(CH₂)ₜ₇₃—NH₂; R⁷³ and R⁷⁶ are, independently, —S—(CH₂)ₜ₇₄—NH₂, —(CH₂)ₜ₇₅—NH₂, or —O—(CH₂)ₜ₇₆—NH₂; t71 and t74 are independently 2 or 3; t72 and t75 are independently 2 or 3; and t73 and t76 are independently 2 or 3.

e) A method of inhibiting tumor growth comprising contacting said tumor with an effective amount of a compound or pharmaceutically acceptable salt thereof; wherein the compound or pharmaceutically acceptable salt is a compound of Formula I:

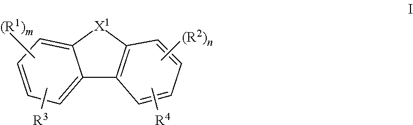

or pharmaceutically acceptable salt thereof, wherein: X¹ is O, S, S(═O), or S(═O)₂; R¹ and R² are, independently, halo, C₁₋₄ alkyl, C₁₋₄ alkoxy, CN, C₁₋₄ haloalkyl, or C₁₋₄ haloalkoxy; R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶; each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸; each R⁶ is independently, halo, OH, C₁₋₄ alkyl, C₁₋₄ alkoxy, CN, C₁₋₄ haloalkyl, or C₁₋₄ haloalkoxy; R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl; m is 0 or 1; n is 0 or 1; each t1 is independently 2 or 3; each t2 is independently 1, 2, or 3; each t3 is independently 2 or 3; each t4 is independently 2 or 3; and each t5 is independently 2 or 3; or the compound or pharmaceutically acceptable salt is a compound of Formula II:

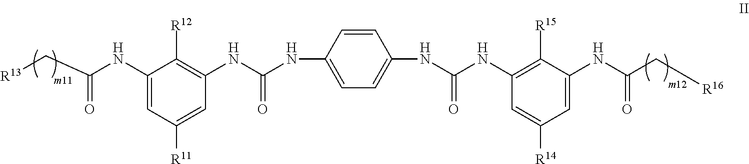

or pharmaceutically acceptable salt thereof, wherein: R¹¹ and R¹⁴ are, independently, H, Cl, CN, methyl, CH₂F, CHF₂, or CF₃; R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂; R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂; each R¹⁷ is independently, pyrrolidinyl, piperidinyl, morpholinyl, or piperazinyl, each optionally substituted with 1 or 2 C₁₋₄ alkyl; each of t11, t12, t13, and t14 is independently 2 or 3; each of m11 and m12 is independently 3, 4, or 5; each of m13 and m14 is independently 1, 2, 3, 4, or 5; or the compound or pharmaceutically acceptable salt is a compound of Formula III:

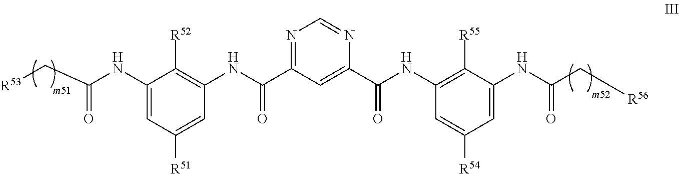

or pharmaceutically acceptable salt thereof, wherein: R⁵¹ and R⁵⁴ are, independently, H, Cl, CN, methyl, CH₂F, CHF₂, or CF₃; R⁵² and R⁵⁵ are, independently, —S—(CH₂)ₜ₅₁—NH₂ or —(CH₂)ₜ₅₂—NH₂; R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂; each of t51 and t52 is independently 2 or 3; each of m51 and m52 is independently 3, 4, or 5; and each of m53 and m54 is independently 1, 2, 3, 4, or 5; or the compound or pharmaceutically acceptable salt is a compound of Formula IV or IVa:

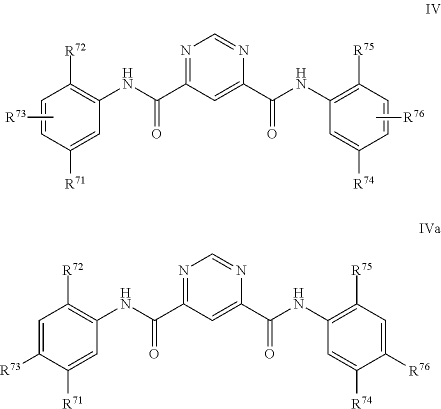

or pharmaceutically acceptable salt thereof, wherein: R⁷¹ and R⁷⁴ are, independently, H, Cl, CN, methyl, CH₂F, CHF₂, or CF₃; R⁷² and R⁷⁵ are, independently, —S—(CH₂)ₜ₇₁′—NH₂, —(CH₂)ₜ₇₂—NH₂, or —O—(CH₂)ₜ₇₃—NH₂; R⁷³ and R⁷⁶ are, independently, —S—(CH₂)ₜ₇₄—NH₂, —(CH₂)ₜ₇₅—NH₂, or —O—(CH₂)ₜ₇₆—NH₂; t71 and t74 are independently 2 or 3; t72 and t75 are independently 2 or 3; and t73 and t76 are independently 2 or 3.

f) A method of treating or preventing spread or metastasis of cancer in an animal comprising administering to said animal an effective amount of a compound or pharmaceutically acceptable salt thereof; wherein the compound or pharmaceutically acceptable salt administered is a compound of Formula I:

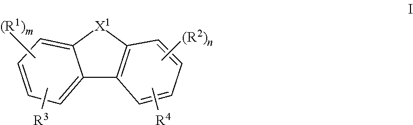

or pharmaceutically acceptable salt thereof, wherein: X¹ is O, S, S(═O), or S(═O)₂; R¹ and R² are, independently, halo, C₁₋₄ alkyl, C₁₋₄ alkoxy, CN, C₁₋₄ haloalkyl, or C₁₋₄ haloalkoxy; R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶; each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂₅—S—(CH₂)ₜ₅—NH₂, —O—(CH₂)ₜ₆—NH₂, or NR⁷R⁸; each R⁶ is independently, halo, OH, C₁₋₄ alkyl, C₁₋₄ alkoxy, CN, C₁₋₄ haloalkyl, or C₁₋₄ haloalkoxy; R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl; m is 0 or 1; n is 0 or 1; each t1 is independently 2 or 3; each t2 is independently 1, 2, or 3; each t3 is independently 2 or 3; each t4 is independently 2 or 3; and each t5 is independently 2 or 3; or the compound or pharmaceutically acceptable salt administered is a compound of Formula II:

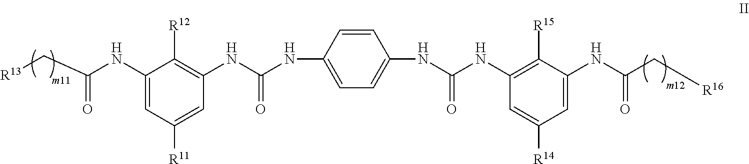

or pharmaceutically acceptable salt thereof, wherein: R¹¹ and R¹⁴ are, independently, H, Cl, CN, methyl, CH₂F, CHF₂, or CF₃; R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂; R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂; each R¹⁷ is independently, pyrrolidinyl, piperidinyl, morpholinyl, or piperazinyl, each optionally substituted with 1 or 2 C₁₋₄ alkyl; each of t11, t12, t13, and t14 is independently 2 or 3; each of m11 and m12 is independently 3, 4, or 5; each of m13 and m14 is independently 1, 2, 3, 4, or 5; or the compound or pharmaceutically acceptable salt administered is a compound of Formula III:

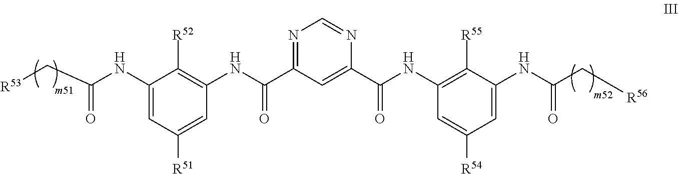

or pharmaceutically acceptable salt thereof, wherein: R⁵¹ and R⁵⁴ are, independently, H, Cl, CN, methyl, CH₂F, CHF₂, or CF₃; R⁵² and R⁵⁵ are, independently, —S—(CH₂)ₜ₅₁—NH₂ or —(CH₂)ₜ₅₂—NH₂; R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂; each of t51 and t52 is independently 2 or 3; each of m51 and m52 is independently 3, 4, or 5; and each of m53 and m54 is independently 1, 2, 3, 4, or 5; or the compound or pharmaceutically acceptable salt administered is a compound of Formula IV or IVa:

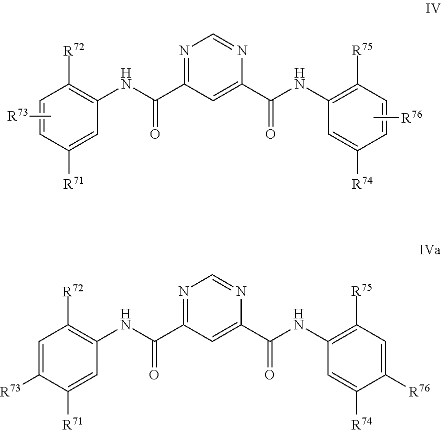

or pharmaceutically acceptable salt thereof, wherein: R⁷¹ and R⁷⁴ are, independently, H, Cl, CN, methyl, CH₂F, CHF₂, or CF₃; R⁷² and R⁷⁵ are, independently, —S—(CH₂)ₜ₇₁′—NH₂, —(CH₂)ₜ₇₂—NH₂, or —O—(CH₂)ₜ₇₃—NH₂; R⁷³ and R⁷⁶ are, independently, —S—(CH₂)ₜ₇₄—NH₂, —(CH₂)ₜ₇₅—NH₂, or —O—(CH₂)ₜ₇₆—NH₂; t71 and t74 are independently 2 or 3; t72 and t75 are independently 2 or 3; and t73 and t76 are independently 2 or 3.

g) A method of treating an animal afflicted with a tumor or cancer comprising administering to said animal an effective amount of a compound or pharmaceutically acceptable salt thereof; wherein the compound or pharmaceutically acceptable salt administered is a compound of Formula I:

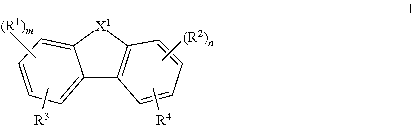

or pharmaceutically acceptable salt thereof, wherein: X¹ is O, S, S(═O), or S(═O)₂; R¹ and R² are, independently, halo, C₁₋₄ alkyl, C₁₋₄ alkoxy, CN, C₁₋₄ haloalkyl, or C₁₋₄ haloalkoxy; R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶; each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂₅—S—(CH₂)ₜ₅—NH₂, —O—(CH₂)ₜ₆—NH₂, or NR⁷R⁸; each R⁶ is independently, halo, OH, C₁₋₄ alkyl, C₁₋₄ alkoxy, CN, C₁₋₄ haloalkyl, or C₁₋₄ haloalkoxy; R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl; m is 0 or 1; n is 0 or 1; each t1 is independently 2 or 3; each t2 is independently 1, 2, or 3; each t3 is independently 2 or 3; each t4 is independently 2 or 3; and each t5 is independently 2 or 3; or the compound or pharmaceutically acceptable salt administered is a compound of Formula II:

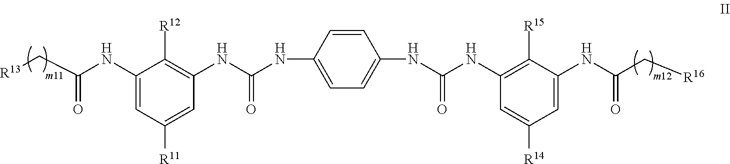

or pharmaceutically acceptable salt thereof, wherein: R¹¹ and R¹⁴ are, independently, H, Cl, CN, methyl, CH₂F, CHF₂, or CF₃; R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂; R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂; each R¹⁷ is independently, pyrrolidinyl, piperidinyl, morpholinyl, or piperazinyl, each optionally substituted with 1 or 2 C₁₋₄ alkyl; each of t11, t12, t13, and t14 is independently 2 or 3; each of m11 and m12 is independently 3, 4, or 5; each of m13 and m14 is independently 1, 2, 3, 4, or 5; or the compound or pharmaceutically acceptable salt administered is a compound of Formula III:

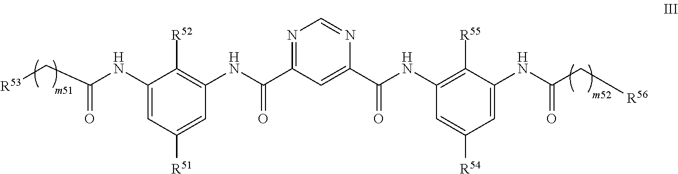

or pharmaceutically acceptable salt thereof, wherein: R⁵¹ and R⁵⁴ are, independently, H, Cl, CN, methyl, CH₂F, CHF₂, or CF₃; R⁵² and R⁵⁵ are, independently, —S—(CH₂)ₜ₅₁—NH₂ or —(CH₂)ₜ₅₂—NH₂; R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂; each of t51 and t52 is independently 2 or 3; each of m51 and m52 is independently 3, 4, or 5; and each of m53 and m54 is independently 1, 2, 3, 4, or 5; or the compound or pharmaceutically acceptable salt administered is a compound of Formula IV or IVa:

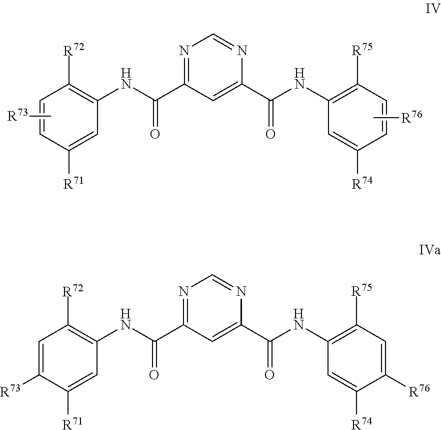

or pharmaceutically acceptable salt thereof, wherein: R⁷¹ and R⁷⁴ are, independently, H, Cl, CN, methyl, CH₂F, CHF₂, or CF₃; R⁷² and R⁷⁵ are, independently, —S—(CH₂)ₜ₇₁′—NH₂, —(CH₂)ₜ₇₂—NH₂, or —O—(CH₂)ₜ₇₃—NH₂; R⁷³ and R⁷⁶ are, independently, —S—(CH₂)ₜ₇₄—NH₂, —(CH₂)ₜ₇₅—NH₂, or —O—(CH₂)ₜ₇₆—NH₂; t71 and t74 are independently 2 or 3; t72 and t75 are independently 2 or 3; and t73 and t76 are independently 2 or 3.

### DESCRIPTION OF THE INVENTION

Unless otherwise defined, the terms below have the following meanings

The terms “treat,” “treated,” or “treating” as used herein refers to both therapeutic treatment and prophylactic or preventative measures wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; or enhancement or improvement of condition, disorder or disease. Treatment includes eliciting a clinically significant response without excessive levels of side effects. Treatment also includes prolonging survival as compared to expected survival if not receiving treatment.

The term “animal” as used herein includes, but is not limited to, humans and non-human vertebrates such as wild, domestic and farm animals.

The term “amphiphilic” as used herein describes a three-dimensional structure having discrete hydrophobic and hydrophilic regions. An amphiphilic compound suitably has the presence of both hydrophobic and hydrophilic elements.

The term “facially amphiphilic” or “facial amphiphilicity” as used herein describes compounds with polar (hydrophilic) and nonpolar (hydrophobic) side chains that adopt conformation(s) leading to segregation of polar and nonpolar side chains to opposite faces or separate regions of the structure or molecule.

The phrase “groups with chemically nonequivalent termini” refers to functional groups such as esters amides, sulfonamides and N-hydroxyoximes where reversing the orientation of the substituents, e.g. R¹C(═O)OR² vs. R¹O(O═)CR², produces unique chemical entities.

The phrase “in need thereof” as used herein means that the animal or mammal has been identified as having a need for the particular method or treatment. In some embodiments, the identification can be by any means of diagnosis. In any of the methods and treatments described herein, the animal or mammal can be in need thereof.

The term “alkyl” as used herein by itself or as part of another group refers to both straight and branched chain radicals from 1 to 12 carbons, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl.

The term “alkenyl” as used herein refers to a straight or branched chain radical of 2-20 carbon atoms, unless the chain length is limited thereto, including, but not limited to, ethenyl, 1-propenyl, 2-propenyl, 2-methyl-1-propenyl, 1-butenyl, 2-butenyl, and the like. Suitably, the alkenyl chain is from 2 to 10 carbon atoms in length, or from 2 to 8 carbon atoms in length, or from 2 to 4 carbon atoms in length.

The term “alkynyl” as used herein refers to a straight or branched chain radical of 2-20 carbon atoms, unless the chain length is limited thereto, wherein there is at least one triple bond between two of the carbon atoms in the chain, including, but not limited to, acetylene, 1-propylene, 2-propylene, and the like. Suitably, the alkynyl chain is 2 to 10 carbon atoms in length, or from 2 to 8 carbon atoms in length, or from 2 to 4 carbon atoms in length.

The term “alkylene” as used herein refers to an alkyl linking group, i.e., an alkyl group that links one group to another group in a molecule.

The term “alkoxy” as used herein refers to mean a straight or branched chain radical of 1 to 20 carbon atoms, unless the chain length is limited thereto, bonded to an oxygen atom, including, but not limited to, methoxy, ethoxy, n-propoxy, isopropoxy, and the like. Suitably, the alkoxy chain is from 1 to 10 carbon atoms in length, or from 1 to 8 carbon atoms in length, or from 1 to 6 carbon atoms in length.

The term “aryl” as used herein by itself or as part of another group refers to monocyclic or bicyclic aromatic groups containing from 6 to 12 carbons in the ring portion, preferably 6-10 carbons in the ring portion, such as the carbocyclic groups phenyl, naphthyl or tetrahydronaphthyl. The term “aryl” can represent carbocyclic aryl groups, such as phenyl, naphthyl or tetrahydronaphthyl, as well as heterocyclic aryl (“heteroaryl”) groups, such as pyridyl, pyrimidinyl, pyridazinyl, furyl, and pyranyl.

The term “arylene” as used herein by itself or as part of another group refers to an aryl linking group, i.e., an aryl group that links one group to another group in a molecule.

The term “cycloalkyl” as used herein by itself or as part of another group refers to cycloalkyl groups containing from 3 to 9 carbon atoms, or from 3 to 8 carbon atoms. Typical examples are cyclopropyl, cyclobutyl, cyclopentyl, cyclohexyl, cycloheptyl, cyclooctyl and cyclononyl.

The term “halogen” or “halo” as used herein by itself or as part of another group refers to chlorine, bromine, fluorine or iodine.

The term “hydroxy” or “hydroxyl” as used herein by itself or as part of another group refers to an —OH group.

The term “heteroaryl” as used herein refers to groups having 5 to 14 ring atoms; 6, 10 or 14 π-electrons shared in a cyclic array; and containing carbon atoms and 1, 2 or 3 oxygen, nitrogen or sulfur heteroatoms. Examples of heteroaryl groups include thienyl, imadizolyl, oxadiazolyl, isoxazolyl, triazolyl, pyridyl, pyrimidinyl, pyridazinyl, furyl, pyranyl, thianthrenyl, pyrazolyl, pyrazinyl, indolizinyl, isoindolyl, isobenzofuranyl, benzoxazolyl, xanthenyl, 2H-pyrrolyl, pyrrolyl, 3H-indolyl, indolyl, indazolyl, purinyl, 4H-quinolizinyl, isoquinolyl, quinolyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furazanyl, and phenoxazinyl groups. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

The term “heteroarylene” as used herein by itself or as part of another group refers to a heteroaryl linking group, i.e., a heteroaryl group that links one group to another group in a molecule.

The term “heterocycle” or “heterocyclic ring”, as used herein except where noted, represents a stable 5- to 7-membered mono- or bicyclic or stable 7- to 10-membered bicyclic heterocyclic ring system any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Especially useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of such heterocyclic groups include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

The term “alkylamino” as used herein by itself or as part of another group refers to an amino group which is substituted with one alkyl group having from 1 to 6 carbon atoms. The term “dialkylamino” as used herein by itself or as part of an other group refers to an amino group which is substituted with two alkyl groups, each having from 1 to 6 carbon atoms.

The term “alkylthio” as used herein by itself or as part of an other group refers to a thio group which is substituted with one alkyl group having from 1 to 6 carbon atoms.

The term “lower acylamino” as used herein by itself or as part of an other group refers to an amino group substituted with a C₁-C₆ alkylcarbonyl group.

The term “chemically nonequivalent termini” as used herein refers to a functional group such as an ester, amide, sulfonamide, or N-hydroxyoxime that, when reversing the orientation of the functional group (e.g., —(C═O)O—) produces different chemical entities (e.g., —R¹C(═O)OR²— vs. —R¹OC(═O)R²—).

The term “treatment of cancer” or “treating cancer” refers to the prevention or alleviation or amelioration of any of the specific phenomena known in the art to be associated with the pathology commonly known as “cancer.” The term “cancer” refers to the spectrum of pathological symptoms associated with the initiation or progression, as well as metastasis, of malignant tumors. By the term “tumor” is intended, for the purpose of the present invention, a new growth of tissue in which the multiplication of cells is uncontrolled and progressive. The tumor that is particularly relevant to the invention is the malignant tumor, one in which the primary tumor has the properties of invasion or metastasis or which shows a greater degree of anaplasia than do benign tumors. Thus, “treatment of cancer” or “treating cancer” refers to an activity that prevents, alleviates or ameliorates any of the primary phenomena (initiation, progression, metastasis) or secondary symptoms associated with the disease.

The present invention provides non-peptidic, facially amphiphilic compounds, pharmaceutical compositions of the same, and methods of using the same to treat or reduce cancer.

The compounds of the present invention are capable of adopting amphiphilic conformations that allow for the segregation of polar and nonpolar regions of the molecule into different spatial regions.

The facially amphiphilic conformations adopted by the compounds of the present invention form the basis for a number of applications. For example, the compounds possess anti-microbial activity and are useful as anti-microbial agents. Use of the compounds as anti-microbial agents is reported in WIPO Publication No. WO 2004/082634, the contents of which is fully incorporated by reference herein in its entirety. Use of the some of the compounds as anti-microbial agents is reported in U.S. Patent Application Publication No. 2005-0287108, the contents of which is also fully incorporated by reference herein in its entirety.

The compounds also possess anti-cancer or anti-tumorigenic activity and can also be used as anti-cancer and anti-tumor agents, e.g., the compounds can kill or inhibit the growth of cancer cells. Thus, the compounds can be used in methods of treating cancer in an animal. The compounds can also be used in methods of reducing cancer in an animal, or in methods of treating or preventing the spread or metastasis of cancer in an animal, or in methods of treating an animal afflicted with cancer. The compounds can also be used in methods of killing or inhibiting the growth of a cancer cell, or in methods of inhibiting tumor growth. In some embodiments, the compounds of the invention can act directly on the cancer cell rather than by acting indirectly such as by inhibition of angiogenesis.

Many of the compounds are significantly smaller and easier to prepare than their naturally occurring counterparts. They have the same mechanism of action as magainin (a naturally occurring host defense peptide) and are approximately equipotent and as broad in their spectrum of action as magainin. However, the non-peptidic compounds of the present invention are significantly less toxic towards human erythrocytes, much less expensive to prepare, and are expected to be much more stable in vivo.

The compounds of the present invention have been shown to possess anti-tumor or anti-cancer activity. Thus, the compounds of the present invention can be used as anticancer or antineoplastic agents and, for example, can be used in a method of treating cancer in an animal.

The invention is directed to methods of treating cancer in an animal in need thereof, by administering to the animal an effective amount of a pharmaceutical composition comprising a compound of the invention. In some embodiments, one or more compounds may be combined in the same composition for any of the methods disclosed herein.

The present invention provides methods for treating cancer in an animal in need thereof comprising administering to the animal an effective amount of a compound or pharmaceutically acceptable salt thereof; wherein the compound or pharmaceutically acceptable salt administered is a compound of Formula I:

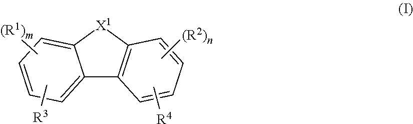

or pharmaceutically acceptable salt thereof, wherein:

X¹ is O, S, S(═O), or S(═O)₂;

R¹ and R² are, independently, halo, C₁₋₄ alkyl, C₁₋₄alkoxy, CN, C₁₋₄haloalkyl, or C₁₋₄ haloalkoxy;

R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶;

each R⁵ is independently —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, —(CH₂)ₜ₇—NH₂, or NR⁷R⁸;

each R⁶ is independently halo, OH, C₁₋₄ alkyl, C₁₋₄alkoxy, CN, C₁₋₄haloalkyl, or C₁₋₄ haloalkoxy;

R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl;

m is 0 or 1;

n is 0 or 1;

each t1 is independently 2 or 3;

each t2 is independently 1, 2, or 3;

each t3 is independently 2 or 3;

each t4 is independently 2 or 3;

each t5 is independently 2 or 3;

each t6 is independently 2 or 3; and

each t7 is independently 2 or 3;

or the compound or pharmaceutically acceptable salt administered is a compound of Formula II:

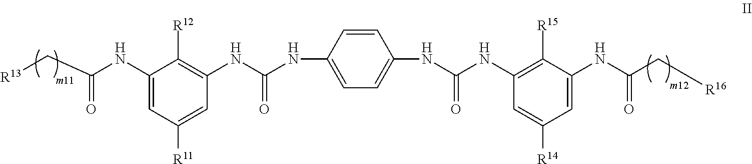

or pharmaceutically acceptable salt thereof, wherein:

R¹¹ and R¹⁴ are, independently, H, Cl, CN, methyl, CH₂F, CHF₂, or CF₃;

R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂;

R¹³ and R¹⁶ are, independently, —NH₂, —NH—(CH₂)ₘ₁₃—NH₂, —NHC(═NH)NH₂, or —NH—(CH₂)ₘ₁₄—NHC(═NH)NH₂;

each R¹⁷ is independently pyrrolidinyl, piperidinyl, morpholinyl, or piperazinyl, each optionally substituted with 1 or 2 C₁₋₄ alkyl;

each of t11, t12, t13, and t14 is independently 2 or 3;

each of m11 and m12 is independently 3, 4, or 5;

each of m13 and m14 is independently 1, 2, 3, 4, or 5;

or the compound or pharmaceutically acceptable salt administered is a compound of Formula III:

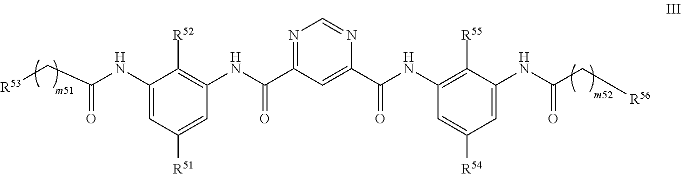

or pharmaceutically acceptable salt thereof, wherein:

R⁵¹ and R⁵⁴ are, independently, H, Cl, CN, methyl, CH₂F, CHF₂, or CF₃;

R⁵² and R⁵⁵ are, independently, —S—(CH₂)ₜ₅₁—NH₂ or —(CH₂)ₜ₅₂—NH₂;

R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂;

each of t51 and t52 is independently 2 or 3;

each of m51 and m52 is independently 3, 4, or 5; and

each of m53 and m54 is independently 1, 2, 3, 4, or 5;

or the compound or pharmaceutically acceptable salt administered is a compound of Formula IV or IVa:

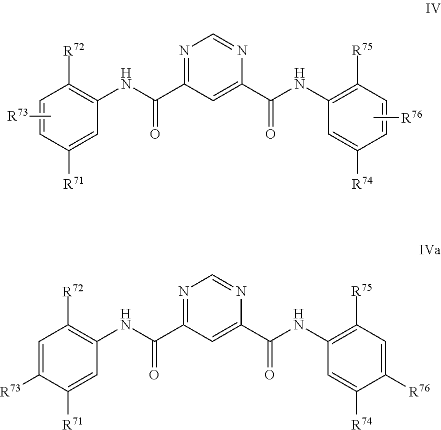

or pharmaceutically acceptable salt thereof, wherein:

R⁷¹ and R⁷⁴ are, independently, H, Cl, CN, methyl, CH₂F, CHF₂, or CF₃;

R⁷² and R⁷⁵ are, independently, —S—(CH₂)ₜ₇₁—NH₂, —(CH₂)ₜ₇₂—NH₂, or —O—(CH₂)ₜ₇₃—NH₂;

R⁷³ and R⁷⁶ are, independently, —S—(CH₂)ₜ₇₄—NH₂, —(CH₂)ₜ₇₅—NH₂, or —O—(CH₂)ₜ₇₆—NH₂;

t71 and t74 are independently 2 or 3;

t72 and t75 are independently 2 or 3; and

t73 and t76 are independently 2 or 3.

In some embodiments, the compound or pharmaceutically acceptable salt thereof administered is a compound of Formula I or pharmaceutically acceptable salt thereof.

In some embodiments, R¹ and R² are, independently, halo, methyl, or C₁ haloalkyl. In some embodiments, R¹ and R² are, independently, Cl, Br, methyl, CH₂F, CHF₂, or CF₃.

In some embodiments, m is 0. In some embodiments, m is 1.

In some embodiments, n is 0. In some embodiments, n is 1.

In some embodiments, R³ and R⁴ are, independently, phenyl or pyridinyl, each substituted with R⁵ and optionally substituted with R⁶. In some embodiments, R³ and R⁴ are, independently, phenyl substituted with R⁵ and optionally substituted with R⁶. In some embodiments, R³ and R⁴ are, independently, pyridinyl substituted with R⁵ and optionally substituted with R⁶.

In some embodiments, each R⁵ is independently —(CH₂)₂—NH₂, —(CH₂)₃—NH₂, —S—(CH₂)₂—NH₂, —O—(CH₂)₃—NH₂, or piperazin-1-yl.

In some embodiments, the compound of Formula I or pharmaceutically acceptable salt thereof is a compound Formula Ia:

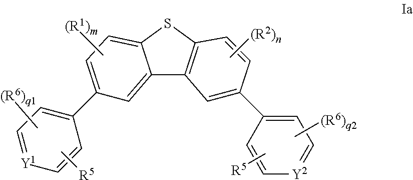

or pharmaceutically acceptable salt thereof, wherein:

R¹ and R² are, independently, Cl, Br, methyl, CH₂F, CHF₂, or CF₃;

each R⁵ is independently —(CH₂)₂—NH₂, —(CH₂)₃—NH₂, —S—(CH₂)₂—NH₂, —O—(CH₂)₃—NH₂, or piperazin-1-yl;

each R⁶ is independently, Cl, Br, methyl, CH₂F, CHF₂, or CF₃;

Y¹ is N or CH;

Y² is N or CH;

m is 0 or 1;

n is 0 or 1;

q1 is 0 or 1; and

q2 is 0 or 1.

In some embodiments, R¹ and R² are, independently, Cl, Br, methyl, or CF₃. In some embodiments, R¹ and R² are, independently, Cl or Br.

In some embodiments, wherein m is 0. In some embodiments, n is 0.

In some embodiments, each R⁵ is independently —(CH₂)₂—NH₂, —(CH₂)₃—NH₂, or piperazin-1-yl. In some embodiments, each R⁵ is independently —(CH₂)₃—NH₂ or piperazin-1-yl.

In some embodiments, q1 is 0.

In some embodiments, q2 is 0.

In some embodiments, the compound of Formula Ia or pharmaceutically acceptable salt thereof is a compound Formula Ia-1:

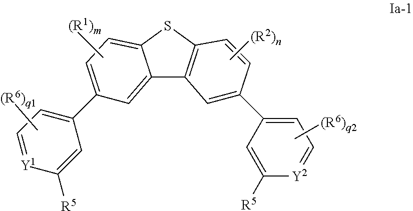

or pharmaceutically acceptable salt thereof.

In some embodiments, the compound of Formula Ia-1 or pharmaceutically acceptable salt thereof is a compound of Formula Ia-1-1:

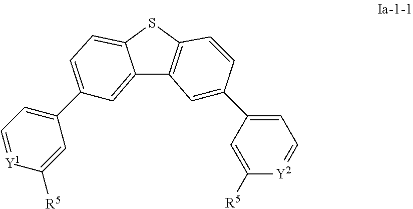

or pharmaceutically acceptable salt thereof.

In some embodiments, the compound of Formula Ia or pharmaceutically acceptable salt thereof is a compound selected from:

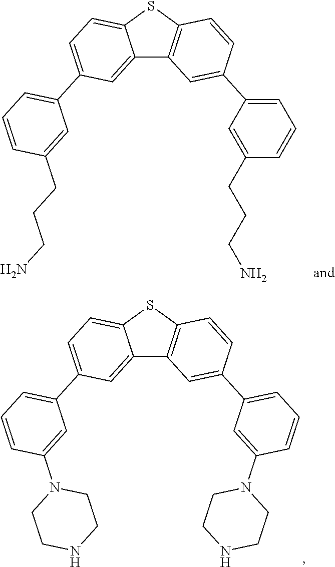

or pharmaceutically acceptable salt thereof.

In some embodiments, the compound of Formula I or pharmaceutically acceptable salt thereof is a compound Formula Ib:

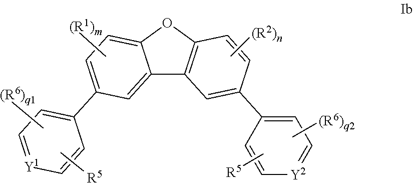

or pharmaceutically acceptable salt thereof, wherein:

R¹ and R² are, independently, Cl, Br, methyl, CH₂F, CHF₂, or CF₃;

each R⁵ is independently —(CH₂)₂—NH₂, —(CH₂)₃—NH₂, —S—(CH₂)₂—NH₂, —O—(CH₂)₃—NH₂, or piperazin-1-yl;

each R⁶ is independently, Cl, Br, methyl, CH₂F, CHF₂, or CF₃;

Y¹ is N or CH;

Y² is N or CH;

m is 0 or 1;

n is 0 or 1;

q1 is 0 or 1; and

q2 is 0 or 1.

In some embodiments, R¹ and R² are, independently, Cl, Br, methyl, or CF₃. In some embodiments, R¹ and R² are, independently, Cl or Br.

In some embodiments, m is 0.

In some embodiments, n is 0.

In some embodiments, each R⁵ is independently —(CH₂)₂—NH₂, —(CH₂)₃—NH₂, or piperazin-1-yl. In some embodiments, each R⁵ is independently —(CH₂)₃—NH₂ or piperazin-1-yl.

In some embodiments, q1 is 0.

In some embodiments, q2 is 0.

In some embodiments, the compound of Formula Ib or pharmaceutically acceptable salt thereof is a compound Formula Ib-1:

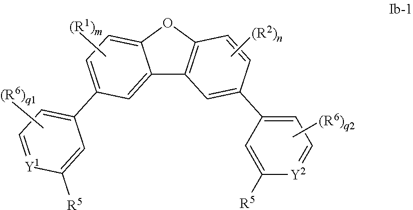

or pharmaceutically acceptable salt thereof.

In some embodiments, the compound of Formula Ib-1 or pharmaceutically acceptable salt thereof is a compound of Formula Ib-1-1:

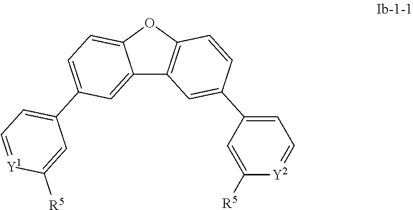

or pharmaceutically acceptable salt thereof.

In some embodiments, the compound of Formula Ib or pharmaceutically acceptable salt thereof is a compound that is

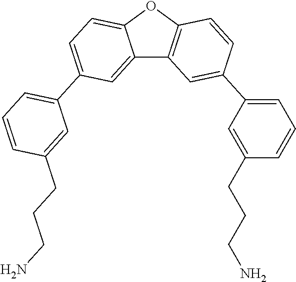

or pharmaceutically acceptable salt thereof.

In some embodiments, the compound or pharmaceutically acceptable salt thereof administered is a compound of Formula II or pharmaceutically acceptable salt thereof.

In some embodiments, R¹¹ and R¹⁴ are, independently, methyl, CH₂F, CHF₂, or CF₃. In some embodiments, R¹¹ and R¹⁴ are, independently, methyl or CF₃. In some embodiments, R¹¹ and R¹⁴ are each CF₃.

In some embodiments, R¹² and R¹⁵ are, independently, —S—(CH₂)₂—NH₂, —O—(CH₂)₂—NH₂, —O—(CH₂)₃—NH₂, —(CH₂)₃—NH₂, or —O—R¹⁷; and each R¹⁷ is independently pyrrolidin-2-yl, pyrrolidin-3-yl, piperidin-2-yl, piperidin-3-yl, piperidin-4-yl, or piperazin-2-yl. In some embodiments, R¹² and R¹⁵ are, independently, —S—(CH₂)₂—NH₂, —O—(CH₂)₃—NH₂, —(CH₂)₃—NH₂, or —O—R¹⁷; and each R¹⁷ is independently pyrrolidin-3-yl or piperidin-3-yl. In some embodiments, R¹² and R¹⁵ are, each —O—R¹⁷; and each R¹⁷ is pyrrolidin-3-yl.

In some embodiments, each of m11 and m12 is 4.

In some embodiments, R¹³ and R¹⁶ are, independently, —NHC(═NH)NH₂ or —NH—(CH₂)ₘ₁₄—NHC(═NH)NH₂; and each m14 is 1, 2, or 3. In some embodiments, R¹³ and R¹¹⁶ are each —NHC(═NH)NH₂.

In some embodiments, the compound of Formula II or pharmaceutically acceptable salt thereof is a compound that is:

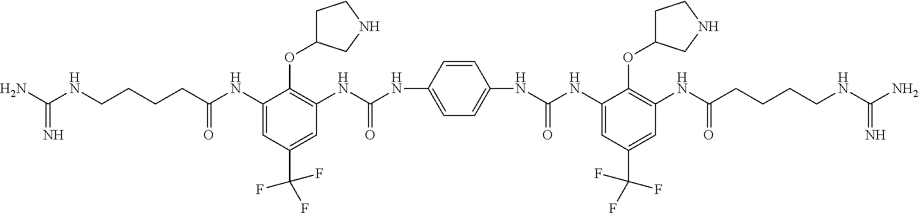

or pharmaceutically acceptable salt thereof.

In some embodiments, the compound or pharmaceutically acceptable salt thereof administered is a compound of Formula III or pharmaceutically acceptable salt thereof.

In some embodiments, R⁵¹ and R⁵⁴ are, independently, methyl, CH₂F, CHF₂, or CF₃. In some embodiments, R⁵¹ and R⁵⁴ are, independently, methyl or CF₃. In some embodiments, R⁵¹ and R⁵⁴ are each CF₃.

In some embodiments, R⁵² and R⁵⁵ are, independently, —S—(CH₂)₂—NH₂ or —(CH₂)₃—NH₂. In some embodiments, R⁵² and R⁵⁵ are each —S—(CH₂)₂—NH₂.

In some embodiments, each of m51 and m52 is 4. In some embodiments, R⁵³ and R⁵⁶ are, independently, —NHC(═NH)NH₂ or —NH—(CH₂)ₘ₅₄—NHC(═NH)NH₂; and each m54 is 1, 2, or 3.

In some embodiments, R⁵³ and R⁵⁶ are each —NHC(═NH)NH₂.

In some embodiments, the compound of Formula III or pharmaceutically acceptable salt thereof is a compound that is:

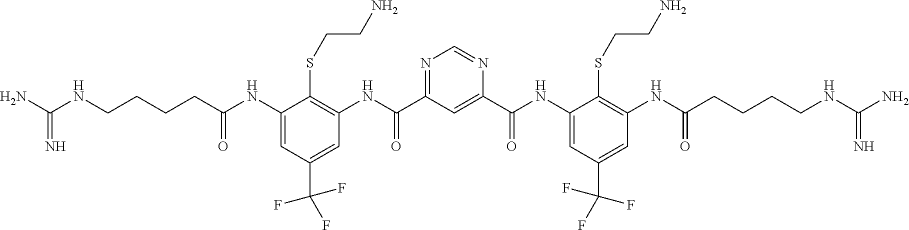

or pharmaceutically acceptable salt thereof.

In some embodiments, the compound or pharmaceutically acceptable salt thereof administered is a compound of Formula IV or pharmaceutically acceptable salt thereof.

In some embodiments, the compound or pharmaceutically acceptable salt thereof administered is a compound of Formula IVa or pharmaceutically acceptable salt thereof.

In some embodiments, R⁷¹ and R⁷⁴ are, independently, methyl, CH₂F, CHF₂, or CF₃. In some embodiments, R⁷¹ and R⁷⁴ are, independently, methyl or CF₃. In some embodiments, R⁷¹ and R⁷⁴ are each CF₃.

In some embodiments, R⁷² and R⁷⁵ are, independently, —S—(CH₂)₂—NH₂, —S(CH₂)₃—NH₂, —(CH₂)₃—NH₂, or —O(CH₂)₃—NH₂. In some embodiments, R⁷² and R⁷⁵ are, independently, —S—(CH₂)₂—NH₂, —(CH₂)₃—NH₂, or —O(CH₂)₃—NH₂. In some embodiments, R⁷² and R⁷⁵ are each —S—(CH₂)₂—NH₂.

In some embodiments, R⁷³ and R⁷⁶ are, independently, —S—(CH₂)₂—NH₂, —S(CH₂)₃—NH₂, —(CH₂)₃—NH₂, or —O(CH₂)₃—NH₂. In some embodiments, R⁷³ and R⁷⁶ are, independently, —S—(CH₂)₂—NH₂, —(CH₂)₃—NH₂, or —O(CH₂)₃—NH₂. In some embodiments, R⁷³ and R⁷⁶ are each —S—(CH₂)₂—NH₂.

In some embodiments, the compound of Formula IV or IVa, or pharmaceutically acceptable salt thereof, is a compound that is:

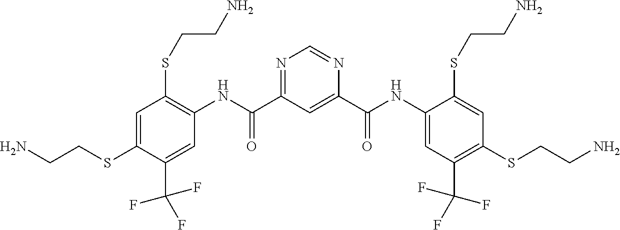

or pharmaceutically acceptable salt thereof.

In some embodiments, the cancer is selected from leukemia, melanoma, lung cancer (such as non-small cell lung cancer), colon cancer, CNS cancer (such as brain cancer), ovary cancer, breast cancer, prostate cancer, and kidney or renal cancer.

The present invention also provides methods for killing or inhibiting growth of a cancer cell comprising contacting the cancer cell with an effective amount of a compound or pharmaceutically acceptable salt thereof; wherein the compound or pharmaceutically acceptable salt is a compound of Formula I:

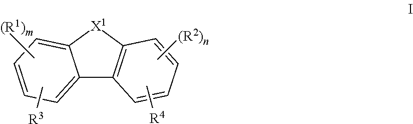

or pharmaceutically acceptable salt thereof, wherein:

X¹ is O, S, S(═O), or S(═O)₂;

R¹ and R² are, independently, halo, C₁₋₄ alkyl, C₁₋₄alkoxy, CN, C₁₋₄ haloalkyl, or C₁₋₄ haloalkoxy;

R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶;

each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸;

each R⁶ is independently, halo, OH, C₁₋₄ alkyl, C₁₋₄alkoxy, CN, C₁₋₄ haloalkyl, or C₁₋₄ haloalkoxy;

R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl;

m is 0 or 1;

n is 0 or 1;

each t1 is independently 2 or 3;

each t2 is independently 1, 2, or 3;

each t3 is independently 2 or 3;

each t4 is independently 2 or 3; and

each t5 is independently 2 or 3;

or the compound or pharmaceutically acceptable salt is a compound of Formula II:

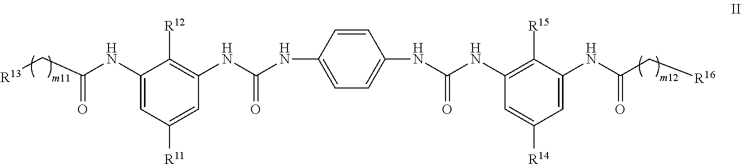

or pharmaceutically acceptable salt thereof, wherein:

R¹¹ and R¹⁴ are, independently, H, Cl, CN, methyl, CH₂F, CHF₂, or CF₃;

R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂;

R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂;

each R¹⁷ is independently, pyrrolidinyl, piperidinyl, morpholinyl, or piperazinyl, each optionally substituted with 1 or 2 C₁₋₄ alkyl;

each of t11, t12, t13, and t14 is independently 2 or 3;

each of m11 and m12 is independently 3, 4, or 5;

each of m13 and m14 is independently 1, 2, 3, 4, or 5;

or the compound or pharmaceutically acceptable salt is a compound of Formula III:

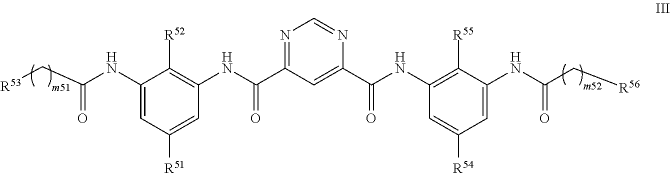

or pharmaceutically acceptable salt thereof, wherein:

R⁵¹ and R⁵⁴ are, independently, H, Cl, CN, methyl, CH₂F, CHF₂, or CF₃;

R⁵² and R⁵⁵ are, independently, —S—(CH₂)ₜ₅₁—NH₂ or —(CH₂)ₜ₅₂—NH₂;

R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂;

each of t51 and t52 is independently 2 or 3;

each of m51 and m52 is independently 3, 4, or 5; and

each of m53 and m54 is independently 1, 2, 3, 4, or 5;

or the compound or pharmaceutically acceptable salt is a compound of Formula IV or IVa:

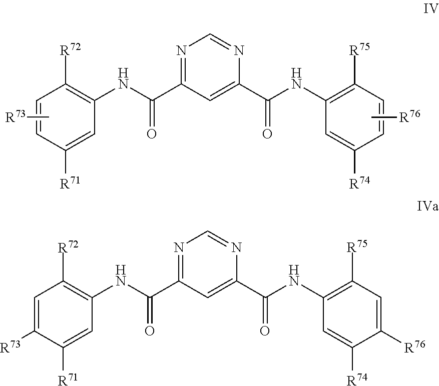

or pharmaceutically acceptable salt thereof, wherein:

R⁷¹ and R⁷⁴ are, independently, H, Cl, CN, methyl, CH₂F, CHF₂, or CF₃;

R⁷² and R⁷⁵ are, independently, —S—(CH₂)ₜ₇₁—NH₂, —(CH₂)ₜ₇₂—NH₂, or —O—(CH₂)ₜ₇₃—NH₂;

R⁷³ and R⁷⁶ are, independently, —S—(CH₂)ₜ₇₄—NH₂, —(CH₂)ₜ₇₅—NH₂, or —O—(CH₂)ₜ₇₆—NH₂;

t71 and t74 are independently 2 or 3;

t72 and t75 are independently 2 or 3; and

t73 and t76 are independently 2 or 3.

The present invention also provides methods for reducing cancer in an animal comprising administering to said animal an effective amount of a compound or pharmaceutically acceptable salt thereof; wherein the compound or pharmaceutically acceptable salt administered is a compound of Formula I:

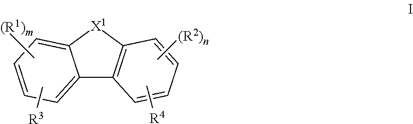

or pharmaceutically acceptable salt thereof, wherein:

X¹ is O, S, S(═O), or S(═O)₂;

R¹ and R² are, independently, halo, C₁₋₄ alkyl, C₁₋₄alkoxy, CN, C₁₋₄haloalkyl, or C₁₋₄haloalkoxy;

R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶;

each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸;

each R⁶ is independently, halo, OH, C₁₋₄ alkyl, C₁₋₄alkoxy, CN, C₁₋₄haloalkyl, or C₁₋₄ haloalkoxy;

R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl;

m is 0 or 1;

n is 0 or 1;

each t1 is independently 2 or 3;

each t2 is independently 1, 2, or 3;

each t3 is independently 2 or 3;

each t4 is independently 2 or 3; and

each t5 is independently 2 or 3;

or the compound or pharmaceutically acceptable salt administered is a compound of Formula II:

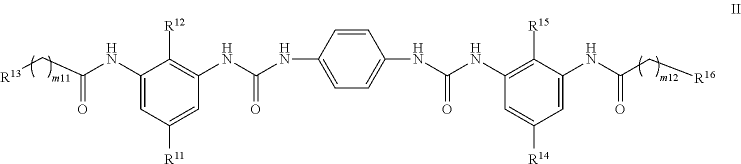

or pharmaceutically acceptable salt thereof, wherein:

R¹¹ and R¹⁴ are, independently, H, Cl, CN, methyl, CH₂F, CHF₂, or CF₃;

R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂;

R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂;

each R¹⁷ is independently, pyrrolidinyl, piperidinyl, morpholinyl, or piperazinyl, each optionally substituted with 1 or 2 C₁₋₄ alkyl;

each of t11, t12, t13, and t14 is independently 2 or 3;

each of m11 and m12 is independently 3, 4, or 5;

each of m13 and m14 is independently 1, 2, 3, 4, or 5;

or the compound or pharmaceutically acceptable salt administered is a compound of Formula III:

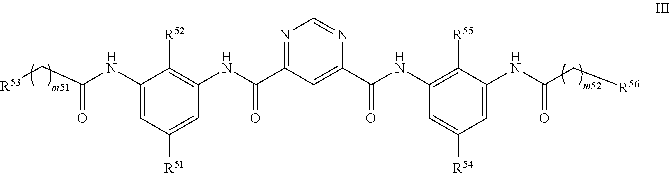

or pharmaceutically acceptable salt thereof, wherein:

R⁵¹ and R⁵⁴ are, independently, H, Cl, CN, methyl, CH₂F, CHF₂, or CF₃;

R⁵² and R⁵⁵ are, independently, —S—(CH₂)ₜ₅₁—NH₂ or —(CH₂)ₜ₅₂—NH₂;

R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂;

each of t51 and t52 is independently 2 or 3;

each of m51 and m52 is independently 3, 4, or 5; and

each of m53 and m54 is independently 1, 2, 3, 4, or 5;

or the compound or pharmaceutically acceptable salt administered is a compound of Formula IV or IVa:

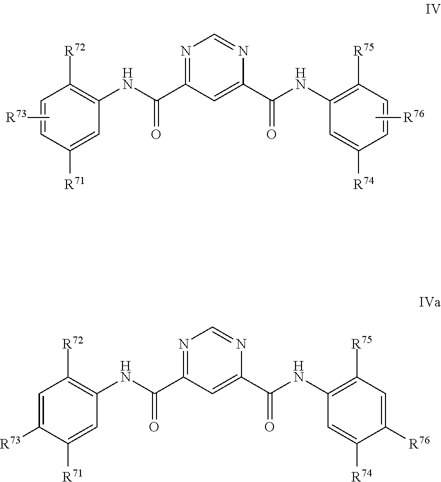

or pharmaceutically acceptable salt thereof, wherein:

R⁷¹ and R⁷⁴ are, independently, H, Cl, CN, methyl, CH₂F, CHF₂, or CF₃;

R⁷² and R⁷⁵ are, independently, —S—(CH₂)ₜ₇₁—NH₂, —(CH₂)ₜ₇₂—NH₂, or —O—(CH₂)ₜ₇₃—NH₂;

R⁷³ and R⁷⁶ are, independently, —S—(CH₂)ₜ₇₄—NH₂, —(CH₂)ₜ₇₅—NH₂, or —O—(CH₂)ₜ₇₆—NH₂;

t71 and t74 are independently 2 or 3;

t72 and t75 are independently 2 or 3; and

t73 and t76 are independently 2 or 3.

The present invention also provides methods of reducing cancer in an animal comprising administering to said animal an effective amount of a compound or pharmaceutically acceptable salt thereof; wherein the compound or pharmaceutically acceptable salt administered is a compound of Formula I:

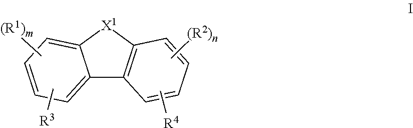

or pharmaceutically acceptable salt thereof, wherein:

X¹ is O, S, S(═O), or S(═O)₂;

R¹ and R² are, independently, halo, C₁₋₄ alkyl, C₁₋₄ alkoxy, CN, C₁₋₄ haloalkyl, or C₁₋₄ haloalkoxy;

R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶;

each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸;

each R⁶ is independently, halo, OH, C₁₋₄ alkyl, C₁₋₄ alkoxy, CN, C₁₋₄ haloalkyl, or C₁₋₄ haloalkoxy;

R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl;

m is 0 or 1;

n is 0 or 1;

each t1 is independently 2 or 3;

each t2 is independently 1, 2, or 3;

each t3 is independently 2 or 3;

each t4 is independently 2 or 3; and

each t5 is independently 2 or 3;

or the compound or pharmaceutically acceptable salt administered is a compound of Formula II:

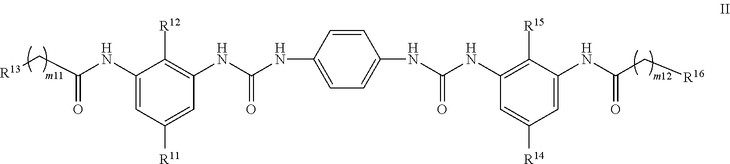

or pharmaceutically acceptable salt thereof, wherein:

R¹¹ and R¹⁴ are, independently, H, Cl, CN, methyl, CH₂F, CHF₂, or CF₃;

R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂;

R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂;

each R¹⁷ is independently, pyrrolidinyl, piperidinyl, morpholinyl, or piperazinyl, each optionally substituted with 1 or 2 C₁₋₄ alkyl;

each of t11, t12, t13, and t14 is independently 2 or 3;

each of m11 and m12 is independently 3, 4, or 5;

each of m13 and m14 is independently 1, 2, 3, 4, or 5;

or the compound or pharmaceutically acceptable salt administered is a compound of Formula III:

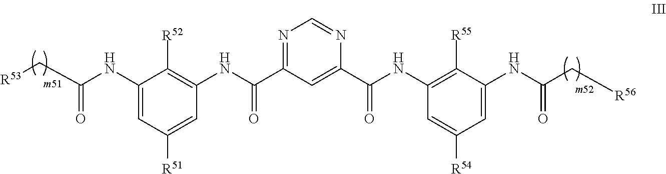

or pharmaceutically acceptable salt thereof, wherein:

R⁵¹ and R⁵⁴ are, independently, H, Cl, CN, methyl, CH₂F, CHF₂, or CF₃;

R⁵² and R⁵⁵ are, independently, —S—(CH₂)ₜ₅₁—NH₂ or —(CH₂)ₜ₅₂—NH₂;

R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂;

each of t51 and t52 is independently 2 or 3;

each of m51 and m52 is independently 3, 4, or 5; and

each of m53 and m54 is independently 1, 2, 3, 4, or 5;

or the compound or pharmaceutically acceptable salt administered is a compound of Formula IV or IVa:

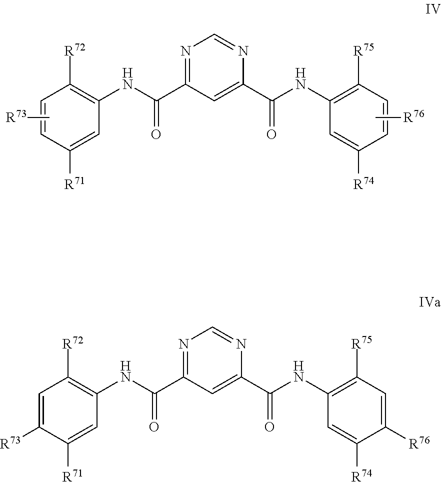

or pharmaceutically acceptable salt thereof, wherein:

R⁷¹ and R⁷⁴ are, independently, H, Cl, CN, methyl, CH₂F, CHF₂, or CF₃;

R⁷² and R⁷⁵ are, independently, —S—(CH₂)ₜ₇₁—NH₂, —(CH₂)ₜ₇₂—NH₂, or —O—(CH₂)ₜ₇₃NH₂;

R⁷³ and R⁷⁶ are, independently, —S—(CH₂)ₜ₇₄—NH₂, —(CH₂)ₜ₇₅—NH₂, or —O—(CH₂)ₜ₇₆—NH₂;

t71 and t74 are independently 2 or 3;

t72 and t75 are independently 2 or 3; and

t73 and t76 are independently 2 or 3.

The present invention also provides methods of inhibiting tumor growth comprising contacting said tumor with an effective amount of a compound or pharmaceutically acceptable salt thereof; wherein the compound or pharmaceutically acceptable salt is a compound of Formula I:

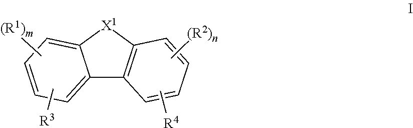

or pharmaceutically acceptable salt thereof, wherein:

X¹ is O, S, S(═O), or S(═O)₂;

R¹ and R² are, independently, halo, C₁₋₄ alkyl, C₁₋₄ alkoxy, CN, C₁₋₄ haloalkyl, or C₁₋₄ haloalkoxy;

R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶;

each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸;

each R⁶ is independently, halo, OH, C₁₋₄ alkyl, C₁₋₄ alkoxy, CN, C₁₋₄ haloalkyl, or C₁₋₄ haloalkoxy;

R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl;

m is 0 or 1;

n is 0 or 1;

each t1 is independently 2 or 3;

each t2 is independently 1, 2, or 3;

each t3 is independently 2 or 3;

each t4 is independently 2 or 3; and

each t5 is independently 2 or 3;

or the compound or pharmaceutically acceptable salt is a compound of Formula II:

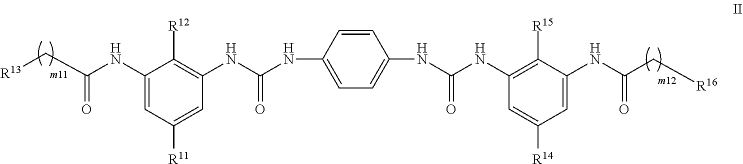

or pharmaceutically acceptable salt thereof, wherein:

R¹¹ and R¹⁴ are, independently, H, Cl, CN, methyl, CH₂F, CHF₂, or CF₃;

R¹² and R¹⁵ are, independently, —S—(CH₂)ₜᵢᵢ—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂;

R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂;

each R¹⁷ is independently, pyrrolidinyl, piperidinyl, morpholinyl, or piperazinyl, each optionally substituted with 1 or 2 C₁₋₄ alkyl;

each of t11, t12, t13, and t14 is independently 2 or 3;

each of m11 and m12 is independently 3, 4, or 5;

each of m13 and m14 is independently 1, 2, 3, 4, or 5;

or the compound or pharmaceutically acceptable salt is a compound of Formula III:

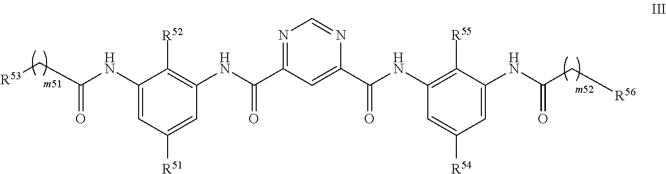

or pharmaceutically acceptable salt thereof, wherein:

R⁵¹ and R⁵⁴ are, independently, H, Cl, CN, methyl, CH₂F, CHF₂, or CF₃;

R⁵² and R⁵⁵ are, independently, —S—(CH₂)ₜ₅₁—NH₂ or —(CH₂)ₜ₅₂—NH₂;

R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂;

each of t51 and t52 is independently 2 or 3;

each of m51 and m52 is independently 3, 4, or 5; and

each of m53 and m54 is independently 1, 2, 3, 4, or 5;

or the compound or pharmaceutically acceptable salt is a compound of Formula IV or IVa:

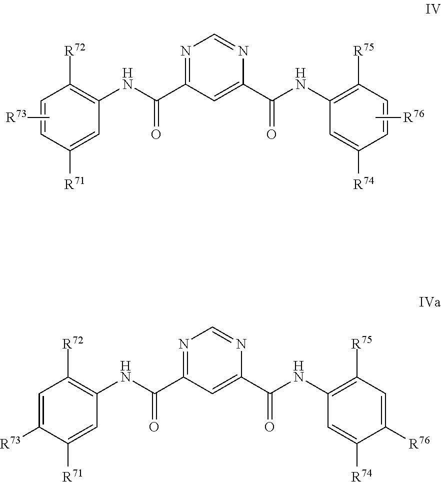

or pharmaceutically acceptable salt thereof, wherein:

R⁷¹ and R⁷⁴ are, independently, H, Cl, CN, methyl, CH₂F, CHF₂, or CF₃;

R⁷² and R⁷⁵ are, independently, —S—(CH₂)ₜ₇₁—NH₂, —(CH₂)ₜ₇₂—NH₂, or —O—(CH₂)ₜ₇₃—NH₂;

R⁷³ and R⁷⁶ are, independently, —S—(CH₂)ₜ₇₄—NH₂, —(CH₂)ₜ₇₅—NH₂, or —O—(CH₂)ₜ₇₆—NH₂;

t71 and t74 are independently 2 or 3;

t72 and t75 are independently 2 or 3; and

t73 and t76 are independently 2 or 3.

The present invention also provides methods of treating or preventing spread or metastasis of cancer in an animal comprising administering to said animal an effective amount of a compound or pharmaceutically acceptable salt thereof; wherein the compound or pharmaceutically acceptable salt administered is a compound of Formula I:

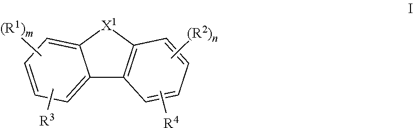

or pharmaceutically acceptable salt thereof, wherein:

X¹ is O, S, S(═O), or S(═O)₂;

R¹ and R² are, independently, halo, C₁₋₄ alkyl, C₁₋₄ alkoxy, CN, C₁₋₄ haloalkyl, or C₁₋₄ haloalkoxy;

R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶;

each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸;

each R⁶ is independently, halo, OH, C₁₋₄ alkyl, C₁₋₄ alkoxy, CN, C₁₋₄ haloalkyl, or C₁₋₄ haloalkoxy;

R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl;

m is 0 or 1;

n is 0 or 1;

each t1 is independently 2 or 3;

each t2 is independently 1, 2, or 3;

each t3 is independently 2 or 3;

each t4 is independently 2 or 3; and

each t5 is independently 2 or 3;

or the compound or pharmaceutically acceptable salt administered is a compound of Formula II:

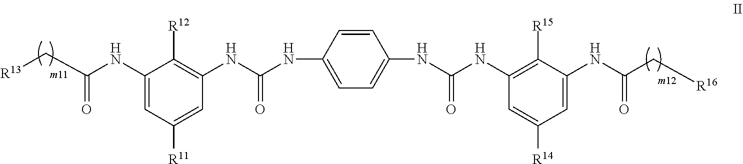

or pharmaceutically acceptable salt thereof, wherein:

R¹¹ and R¹⁴ are, independently, H, Cl, CN, methyl, CH₂F, CHF₂, or CF₃;

R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂;

R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂;

each R¹⁷ is independently, pyrrolidinyl, piperidinyl, morpholinyl, or piperazinyl,

each optionally substituted with 1 or 2 C₁₋₄ alkyl;

each of t11, t12, t13, and t14 is independently 2 or 3;

each of m11 and m12 is independently 3, 4, or 5;

each of m13 and m14 is independently 1, 2, 3, 4, or 5;

or the compound or pharmaceutically acceptable salt administered is a compound of Formula III:

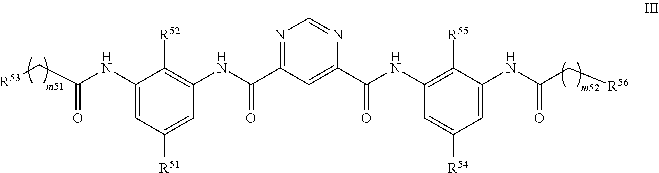

or pharmaceutically acceptable salt thereof, wherein:

R⁵¹ and R⁵⁴ are, independently, H, Cl, CN, methyl, CH₂F, CHF₂, or CF₃;

R⁵² and R⁵⁵ are, independently, —S—(CH₂)ₜ₅₁—NH₂ or —(CH₂)ₜ₅₂—NH₂;

R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂;

each of t51 and t52 is independently 2 or 3;

each of m51 and m52 is independently 3, 4, or 5; and

each of m53 and m54 is independently 1, 2, 3, 4, or 5;

or the compound or pharmaceutically acceptable salt administered is a compound of Formula IV or IVa:

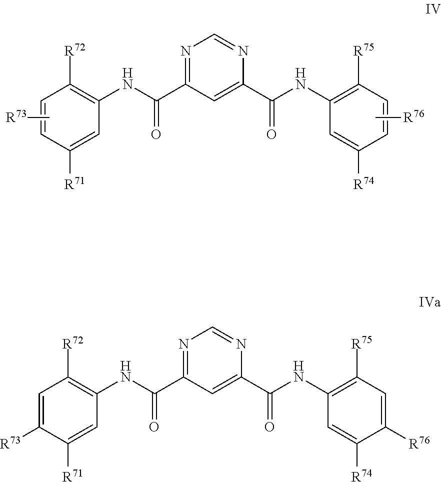

or pharmaceutically acceptable salt thereof, wherein:

R⁷¹ and R⁷⁴ are, independently, H, Cl, CN, methyl, CH₂F, CHF₂, or CF₃;

R⁷² and R⁷⁵ are, independently, —S—(CH₂)ₜ₇₁—NH₂, —(CH₂)ₜ₇₂—NH₂, or —O—(CH₂)ₜ₇₃—NH₂;

R⁷³ and R⁷⁶ are, independently, —S—(CH₂)ₜ₇₄—NH₂, —(CH₂)ₜ₇₅—NH₂, or —O—(CH₂)ₜ₇₆—NH₂;

t71 and t74 are independently 2 or 3;

t72 and t75 are independently 2 or 3; and

t73 and t76 are independently 2 or 3.

The present invention also provides methods of treating an animal afflicted with a tumor or cancer comprising administering to said animal an effective amount of a compound or pharmaceutically acceptable salt thereof; wherein the compound or pharmaceutically acceptable salt administered is a compound of Formula I:

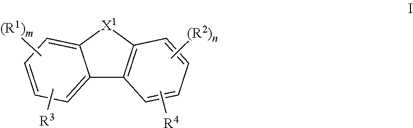

or pharmaceutically acceptable salt thereof, wherein:

X¹ is O, S, S(═O), or S(═O)₂;

R¹ and R² are, independently, halo, C₁₋₄ alkyl, C₁₋₄alkoxy, CN, C₁₋₄haloalkyl, or C₁₋₄haloalkoxy;

R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶;

each R⁵ is independently, —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, or NR⁷R⁸;

each R⁶ is independently, halo, OH, C₁₋₄ alkyl, C₁₋₄alkoxy, CN, C₁₋₄haloalkyl, or C₁₋₄haloalkoxy;

R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl;

m is 0 or 1;

n is 0 or 1;

each t1 is independently 2 or 3;

each t2 is independently 1, 2, or 3;

each t3 is independently 2 or 3;

each t4 is independently 2 or 3; and

each t5 is independently 2 or 3;

or the compound or pharmaceutically acceptable salt administered is a compound of Formula II:

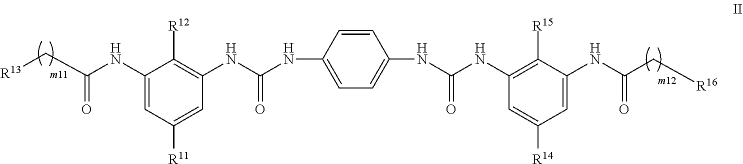

or pharmaceutically acceptable salt thereof, wherein:

R¹¹ and R¹⁴ are, independently, H, Cl, CN, methyl, CH₂F, CHF₂, or CF₃;

R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂;

R¹³ and R¹¹⁶ are, independently, —NH₂, —NH—(CH₂)ₘ₁₃—NH₂, —NHC(═NH)NH₂, or —NH—(CH₂)ₘ₁₄—NHC(═NH)NH₂;

each R¹⁷ is independently, pyrrolidinyl, piperidinyl, morpholinyl, or piperazinyl, each optionally substituted with 1 or 2 C₁₋₄ alkyl;

each of t11, t12, t13, and t14 is independently 2 or 3;

each of m11 and m12 is independently 3, 4, or 5;

each of m13 and m14 is independently 1, 2, 3, 4, or 5;

or the compound or pharmaceutically acceptable salt administered is a compound of Formula III:

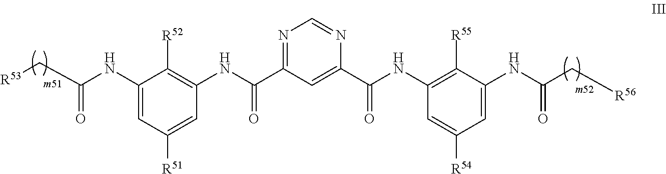

or pharmaceutically acceptable salt thereof, wherein:

R⁵¹ and R⁵⁴ are, independently, H, Cl, CN, methyl, CH₂F, CHF₂, or CF₃;

R⁵² and R⁵⁵ are, independently, —S—(CH₂)ₜ₅₁—NH₂ or —(CH₂)ₜ₅₂—NH₂;

R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂;

each of t51 and t52 is independently 2 or 3;

each of m51 and m52 is independently 3, 4, or 5; and

each of m53 and m54 is independently 1, 2, 3, 4, or 5;

or the compound or pharmaceutically acceptable salt administered is a compound of Formula IV or IVa:

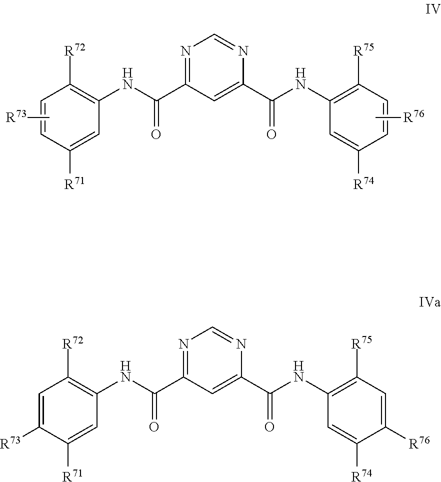

or pharmaceutically acceptable salt thereof, wherein:

R⁷¹ and R⁷⁴ are, independently, H, Cl, CN, methyl, CH₂F, CHF₂, or CF₃;

R⁷² and R⁷⁵ are, independently, —S—(CH₂)ₜ₇₁—NH₂, —(CH₂)ₜ₇₂—NH₂, or —O—(CH₂)ₜ₇₃—NH₂;

R⁷³ and R⁷⁶ are, independently, —S—(CH₂)ₜ₇₄—NH₂, —(CH₂)ₜ₇₅—NH₂, or —O—(CH₂)ₜ₇₆—NH₂;

t71 and t74 are independently 2 or 3;

t72 and t75 are independently 2 or 3; and

t73 and t76 are independently 2 or 3.

In some aspects of the invention, the compounds of the present invention are derivatives referred to as prodrugs. The term “prodrug” denotes a derivative of a known direct acting drug, which derivative has enhanced delivery characteristics and therapeutic value as compared to the drug, and is transformed into the active drug by an enzymatic or chemical process.

When any variable occurs more than one time in any constituent or in any of the compounds recited for any of the general Formulae above, its definition on each occurrence is independent of its definition at every other occurrence. Also, combinations of substituents and/or variables are permissible only if such combinations result in stable compounds.

It is understood that the present invention encompasses the use of stereoisomers, diastereomers and optical isomers of the compounds of the present invention, as well as mixtures thereof, for use in the methods disclosed herein. Additionally, it is understood that stereoisomers, diastereomers and optical isomers of the compounds of the present invention, and mixtures thereof, are within the scope of the invention. By way of non-limiting example, the mixture can be a racemate or the mixture may comprise unequal proportions of one particular stereoisomer over the other. Thus, in some aspects of the invention, the compounds of the invention are provided as mixtures that are racemates. Additionally, the compounds of the invention can be provided as a substantially pure stereoisomers, diastereomers and optical isomers. Thus, in some aspects of the invention, the compounds are provided as substantially pure stereoisomers, diastereomers, or optical isomers.

In another aspect of the invention, the compounds of the invention are provided in the form of an acceptable salt (i.e., a pharmaceutically acceptable salt) for treating microbial infections, killing or inhibiting the growth of a microorganism, and providing an antidote to low molecular weight heparin overdose in an animal. Compound salts can be provided for pharmaceutical use, or as an intermediate in preparing the pharmaceutically desired form of the compound. One compound salt that is considered to be acceptable is the hydrochloride acid addition salt. Hydrochloride acid addition salts are often acceptable salts when the pharmaceutically active agent has an amine group that can be protonated. Since a compound of the invention may be polyionic, such as a polyamine, the acceptable compound salt can be provided in the form of a poly(amine hydrochloride).

The compounds of the invention demonstrated herein possess anti-cancer (e.g., anti-neoplastic) activity and can be used to treat cancer in an animal. The compounds can be used in methods of treating cancer in an animal, in methods of reducing cancer in an animal, in methods of treating or preventing the spread or metastasis of cancer in an animal, or in methods of treating an animal afflicted with a tumor or with cancer.

Cancers that are treatable are broadly divided into the categories of carcinoma, lymphoma and sarcoma. Examples of carcinomas that can be treated by the compounds of the present invention include, but are not limited to: adenocarcinoma, acinic cell adenocarcinoma, adrenal cortical carcinomas, alveoli cell carcinoma, anaplastic carcinoma, basaloid carcinoma, basal cell carcinoma, bronchiolar carcinoma, bronchogenic carcinoma, renaladinol carcinoma, embryonal carcinoma, anometroid carcinoma, fibrolamolar liver cell carcinoma, follicular carcinomas, giant cell carcinomas, hepatocellular carcinoma, intraepidermal carcinoma, intraepithelial carcinoma, leptomanigio carcinoma, medullary carcinoma, melanotic carcinoma, menigual carcinoma, mesometonephric carcinoma, oat cell carcinoma, squamal cell carcinoma, sweat gland carcinoma, transitional cell carcinoma, and tubular cell carcinoma. Sarcomas that can be treated by the compounds of the present invention include, but are not limited to: amelioblastic sarcoma, angiolithic sarcoma, botryoid sarcoma, endometrial stroma sarcoma, ewing sarcoma, fascicular sarcoma, giant cell sarcoma, granulositic sarcoma, immunoblastic sarcoma, juxaccordial osteogenic sarcoma, coppices sarcoma, leukocytic sarcoma (leukemia), lymphatic sarcoma (lympho sarcoma), medullary sarcoma, myeloid sarcoma (granulocitic sarcoma), austiogenci sarcoma, periosteal sarcoma, reticulum cell sarcoma (histiocytic lymphoma), round cell sarcoma, spindle cell sarcoma, synovial sarcoma, and telangiectatic audiogenic sarcoma. Lymphomas that can be treated by the compounds of the present invention include, but are not limited to: Hodgkin's disease and lymphocytic lymphomas, such as Burkitt's lymphoma, NPDL, NML, NH and diffuse lymphomas.

Thus, examples of cancers that can be treated using the compounds of the present invention include, but are not limited to, Hodgkin's disease, non-Hodgkin's lymphomas, acute lymphocytic leukemia, multiple myeloma, breast carcinomas, ovarian carcinomas, lung carcinomas, Wilms' tumor, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, rhabdomyosarcoma, Kaposi's sarcoma, genitourinary carcinomas, thyroid carcinomas, esophageal carcinomas, malignant hypercalcemia, renal cell carcinomas, endometrial carcinomas, polycythemia vera, essential thrombocytosis, adrenal cortex carcinomas, skin cancer, and prostatic carcinomas.

Thus, in some aspects, the present invention is directed to a method of treating cancer in an animal in need thereof, the method comprising administering to the animal an effective amount of a pharmaceutical composition comprising a compound described above, and a pharmaceutically acceptable carrier or diluent.

In some aspects, the present invention is also directed to methods of reducing cancer in an animal comprising administering to the animal an effective amount of a compound described above.

In other aspects, the present invention is directed to methods of treating an animal afflicted with a tumor or cancer comprising administering to the animal an effective amount of a compound described above.

The compounds of the present invention appear to be useful in treating metastases.

Thus, in some aspects, the present invention is directed to methods of treating or preventing the spread or metastasis of cancer in an animal comprising administering to the animal an effective amount of a compound described above.

The compounds of the present invention can also be used in methods of killing or inhibiting the growth of cancer cells, either in vivo or in vitro, or inhibiting the growth of a cancerous tumor.

Thus, in some aspects, the invention is also directed to methods of killing or inhibiting the growth of a cancer cell comprising contacting the cancer cell with an effective amount of a compound described above.

In other aspects, the invention is directed to methods of inhibiting tumor growth comprising contacting the tumor with an effective amount of a compound described above.

The compound in any one of the above methods can be administered to a human subject. Thus, in some aspects of the invention, the compound is administered to a human.

The methods disclosed above also have veterinary applications and can be used to treat non-human vertebrates. Thus, in other aspects of the invention, the compound is administered in any one of the above methods to non-human vertebrates, such as wild, domestic, or farm animals, including, but not limited to, cattle, sheep, goats, pigs, dogs, cats, and poultry such as chicken, turkeys, quail, pigeons, ornamental birds and the like.

Preparation of the Compounds of the Present Invention are Described in Detail in WIPO Publication No. WO 2004/082634, and in U.S. Patent Application Publication No. 2005-0287108, the contents of each of which are fully incorporated by reference herein in its entirety.

Briefly, polyamide and polyester compounds of the present invention can be prepared by typical condensation polymerization and addition polymerization processes. See, for example, G. Odian, Principles of Polymerization, John Wiley & Sons, Third Edition (1991), M. Steven, Polymer Chemistry, Oxford University Press (1999). Most commonly the polyamides are prepared by a) thermal dehydration of amine salts of carboxylic acids, b) reaction of acid chlorides with amines and c) aminolysis of esters. The most common method for the preparation of polyureas is the reaction of diamines with diisocyanates. (Yamaguchi et al., Polym. Bull., 2000, 44, 247). This exothermic reaction can be carried out by solution techniques or by interfacial techniques. One skilled in organic and polymer chemistry will appreciate that the diisocyanate can be replaced with a variety of other bis-acylating agents e.g., phosgene or N,N′-(diimidazolyl)carbonyl, with similar results. Polyurethanes are prepared by comparable techniques using a diisocyanate and a dialcohol or by reaction of a diamine with a bis-chloroformate.

The polyaryl and polyarylalkynyl compounds of the present invention are synthesized according to the procedures outlined in WIPO Publ. No. WO 02/072007 and U.S. Patent Application Publication No. 2005-0287108. The entire contents of both WIPO Publ. No. WO 02/072007 and U.S. Patent Application Publication No. 2005-0287108 are fully incorporated herein by reference.

The syntheses of appropriately substituted monomers in the compounds of the invention are straightforward. Numerous pathways are available to incorporate polar and nonpolar side chains. For example, phenolic groups on the monomer can be alkylated. Alkylation of the commercially available phenol will be accomplished with standard Williamson ether synthesis for the non-polar side chain with ethyl bromide as the alkylating agent. Polar sidechains can be introduced with bifunctional alkylating agents such as BOC—NH(CH₂)₂Br. Alternately, the phenol group can be alkylated to install the desired polar side chain function by employing the Mitsonobu reaction with BOC—NH(CH₂)₂—OH, triphenyl phosphine, and diethyl acetylenedicarboxylate. Standard conditions for reduction of the nitro groups and hydrolysis of the ester afford the amino acid. With the aniline and benzoic acid in hand, coupling can be effected under a variety of conditions. Alternately, the hydroxy group of the (di)nitrophenol can be converted to a leaving group and a functionality introduced under nucleophilic aromatic substitution conditions. Other potential scaffolds that can be prepared with similar sequences are methyl 2-nitro-4-hydroxybenzoate and methyl 2-hydroxy-4-nitrobenzoate.

The compound of the present invention are designed using computer-aided computational techniques, such as de novo design techniques, to embody the amphiphilic properties believed to be important for activity. In general, de novo design of oligomers is done by defining a three-dimensional framework of the backbone assembled from a repeating sequence of monomers using molecular dynamics and quantum force field calculations. Next, side groups are computationally grafted onto the backbone to maximize diversity and maintain drug-like properties. The best combinations of functional groups are then computationally selected to produce a cationic, amphiphilic structures. Representative compounds are synthesized from this selected library to verify structures and test their biological activity. Importantly, novel molecular dynamic and coarse grain modeling programs have been developed for this approach because existing force fields developed for biological molecules, such as peptides, were unreliable in these oligomer applications (Car et al., Phys. Rev. Lett., 1985, 55, 2471-2474; Siepmann et al., Mol. Phys., 1992, 75, 59-70; Martin et al., J. Phys. Chem., 1999, B 103, 4508-4517; Brooks et al., J. Comp. Chem., 1983, 4, 187-217). Several chemical structural series of compounds have been prepared. See, for example, WO 02/100295 A2, the entire contents of which are incorporated herein by reference. The compound of the present invention are prepared in a similar manner (see below).

The general approach is as follows:

1) A backbone that should fold into a given, well-defined three-dimensional structure is defined. Extensive theoretical studies are carried out to demonstrate that the compounds are able to adopt the desired secondary conformation. Model compounds (short oligomers) are prepared for structural analysis of folding by X-ray crystallography.

2) The backbone of the compound is then decorated with appropriate functional groups to endow the compound with the desired facial amphiphilic character.

3) The desired compounds are synthesized, and their biological activities are measured.

4) Biophysical studies are carried out to confirm that the compounds are binding to membranes in the desired conformation and that the mechanism of action is as expected from the design.

5) Based on the findings, structures are redesigned to optimize the potency and selectivity of the compounds, and steps 2-4 are re-iterated.

A goal of this approach is to capture the structural and biological properties of antimicrobial peptides within the framework of traditional compounds that can be prepared by inexpensive condensation reactions.

An example of the design, synthesis, and testing of arylamide compounds, a subgroup of compounds disclosed in the present invention, is presented in Tew et al., Proc. Natl. Acad. Sci. USA, 2002, 99, 5110-5114, the contents of which are fully incorporated by reference herein. See also WIPO Publication No. WO 2004/082634, the contents of which are fully incorporated by reference herein in its entirety. Examples of the design, synthesis, and testing of phenylakynyl compounds is presented in U.S. Patent Application Publication No. 2005-0287108, the contents of which are fully incorporated by reference herein in its entirety.

Compounds of the present invention can be synthesized by solid-phase synthetic procedures well know to those of skill in the art. See, for example, Tew et al., Proc. Natl. Acad. Sci. USA, 2002, 99, 5110-5114). See also, Barany et al., Int. J. Pept. Prot. Res., 1987, 30, 705-739; Solid-phase Synthesis: A Practical Guide, Kates, S. A., and Albericio, F., eds., Marcel Dekker, New York (2000); and Dörwald, F. Z., Organic Synthesis on Solid Phase: Supports, Linkers, Reactions, 2nd Ed., Wiley-VCH, Weinheim (2002).

The compounds of the invention may be converted to a pharmaceutically acceptable salt or solvate thereof, such as an acid addition salt, such as a hydrochloride, hydrobromide, phosphate, acetate, fumarate, maleate, tartrate, citrate, methanesulphonate, or p-toluenesulphonate, or the like.

The compounds of the invention are tested for anti-cancer activity by methods known to those of skill in the art. Examples of anti-cancer assays include, but are not limited to, standard cell viability assays, such as the XTT assay described in Example 1 below, or by metabolic activity assays.

The compounds of the present invention are administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication (e.g., whether the compound is administered to treat a microbial infection, or to provide an antidote for hemorrhagic conditions associated with heparin therapy). The mode of administration can depend on the pathogen or microbe to be targeted. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

The pharmaceutical formulations containing the compounds and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a compound as taught in this invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compound compositions can take the form of, e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds, for example, can be applied to a plaster, or can be applied by transdermal, therapeutic systems that are consequently supplied to the organism.

The pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The amount of compounds of the invention to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The amount of a compound described herein that will be effective in the treatment and/or prevention of cancer will depend on the nature and extent of the cancer, and can be determined by standard clinical techniques. In addition, in vitro or in vivo assays may optionally be employed to help identify optimal dosage ranges. The precise dose to be employed in the compositions will also depend on the route of administration, and the seriousness of the disorder, and should be decided according to the judgment of the practitioner and each patient's circumstances. However, a suitable dosage range for oral administration is, generally, from about 0.001 milligram to about 200 milligrams per kilogram body weight. In some embodiments, the oral dose is from about 0.01 milligram to 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight.

In some embodiments, suitable dosage ranges for intravenous (i.v.) administration are 0.01 mg to 500 mg per kg body weight, 0.1 mg to 100 mg per kg body weight, 1 mg to 50 mg per kg body weight, or 10 mg to 35 mg per kg body weight. Suitable dosage ranges for other modes of administration can be calculated based on the forgoing dosages as known by those skilled in the art. For example, recommended dosages for intradermal, intramuscular, intraperitoneal, subcutaneous, epidural, sublingual, intracerebral, intravaginal, transdermal administration or administration by inhalation are in the range of 0.001 mg to 200 mg per kg of body weight, 0.01 mg to 100 mg per kg of body weight, 0.1 mg to 50 mg per kg of body weight, or 1 mg to 20 mg per kg of body weight. Effective doses may be extrapolated from dose-response curves derived from in vitro or animal model test systems. Such animal models and systems are well known in the art.

The compounds can also be administered in combination with other active ingredients, such as, for example, other anti-cancer or anti-neoplastic agents, or in combination with other cancer therapies other than chemotherapy, such as, for example, surgery or radiotherapy.

The following examples will serve to further typify the nature of this invention but should not be construed as a limitation in the scope thereof, which scope is defined solely by the appended claims.

### EXAMPLES

### Example 1

### Breast Cancer Cells

Compound A (see Table 2 below) was tested for its effectiveness against breast cancer cells. Compound A has an extended backbone structure with the cationic charges separated from the hydrophobic backbone by two methylene units. This compound has demonstrated potent cytotoxicity against a broad spectrum of bacteria, including E. coli D31, B. anthracis ATTC 1099, and S. typhymurium ATTC 29631, within a range of 0.8 to 1.6 μg/mL. Compound A also shows significant selectivity towards bacteria: When Compound A is tested against human red blood cells, 50% lysis (HC₅₀) occurs at a concentration of 75 μg/ml.

Compound A was tested against two human breast cancer cell lines, MCF-7 (ATCC HTB-22) and TMX2-28, and one non-tumorigenic breast cell line, MCF-10A (ATCC CRL-10317). MCF-7 and TMX2-28 cells were grown in DC₅ cell growth media while the MCF-10A cells were grown in MEGM, both supplemented with 5% bovine growth serum. The cells were grown using standard techniques. Cell cultures at 50% confluence were harvested with trypsin, seeded onto sterile 96 well plates at a density of 10,000 cells/well and allowed to grow overnight to 50% confluence. Compound A was then added to the growth medium and allowed to further incubate for 48 hours. Viable cells were quantitated using an XTT assay (purchased from Roche).

The results of the study are presented in Table 1.

TABLE 1
Growth inhibition of cancerous and normal cell types by Compound A.

| | | | Selectivity IC₉₀/IC₉₀ |
|---|---|---|---|
| Cell Line | Cell Type | IC90 | MCF10A |
| MCF-7 | tumorigenic | 6.3 | 2 |
| TMX2-28 | tumorigenic | 6.3 | 2 |
| MCF10A | non-tumorigenic | 12.5 | — |

Cytotoxic activity of Compound A against tumorigenic and non-tumorigenic breast cell lines is shown in Table 1. When tested against the tumorigenic cell lines, MCF-7 and TMX2-28, Compound A maximally inhibits cell growth (IC₉₀) at concentrations of 6.3 μg/ml. Against the non-tumorigenic MCF-10A cells, the IC₉₀ is 12.5 μg/mL. This shows that there is a 2-fold selectivity towards cancerous cells over normal cells.

The results of the study demonstrate that compounds of the present invention are selectively cytotoxic for tumor cells over normal cells.

### Example 2

### Methodology for the NCI-60 DTP Human Tumor Cell Line Screen

Several compounds were tested at single concentrations (10 μM) against 59 different human tumor cell lines, representing leukemia, melanoma and cancers of the lung, colon, brain, ovary, breast, prostate, and kidney (see, Table 3). The human tumor cell lines of the cancer screening panel were grown in RPMI 1640 medium containing 5% fetal bovine serum and 2 mM L-glutamine. For a typical screening experiment, cells were inoculated into 96 well microtiter plates in 100 μL at plating densities ranging from 5,000 to 40,000 cells/well depending on the doubling time of individual cell lines. After cell inoculation, the microtiter plates were incubated at 37° C., 5% CO₂, 95% air and 100% relative humidity for 24 hours prior to addition of the compounds.

After 24 hours, two plates of each cell line were fixed in situ with TCA, to represent a measurement of the cell population for each cell line at the time of drug addition (Tz). Compounds were solubilized in dimethyl sulfoxide at 400-fold the desired final maximum test concentration and stored frozen prior to use. At the time of drug addition, an aliquot of frozen concentrate was thawed and diluted to twice the desired final maximum test concentration with complete medium containing 50 μg/ml gentamicin. Additional four, 10-fold or ½ log serial dilutions were made to provide a total of five compound concentrations plus control. Aliquots of 100 μl of these different drug dilutions were added to the appropriate microtiter wells already containing 100 μl of medium, resulting in the required final compound concentrations.

Following drug addition, the plates were incubated for an additional 48 hours at 37° C., 5% CO₂, 95% air, and 100% relative humidity. For adherent cells, the assay was terminated by the addition of cold TCA. Cells were fixed in situ by the gentle addition of 50 μl of cold 50% (w/v) TCA (final concentration, 10% TCA) and incubated for 60 minutes at 4° C. The supernatant was discarded, and the plates were washed five times with tap water and air dried. Sulforhodamine B (SRB) solution (100 μl) at 0.4% (w/v) in 1% acetic acid was added to each well, and plates were incubated for 10 minutes at room temperature. After staining, unbound dye was removed by washing five times with 1% acetic acid and the plates were air dried. Bound stain was subsequently solubilized with 10 mM trizma base, and the absorbance was read on an automated plate reader at a wavelength of 515 nm. For suspension cells, the methodology was the same except that the assay was terminated by fixing settled cells at the bottom of the wells by gently adding 50 μl of 80% TCA (final concentration, 16% TCA). Using the seven absorbance measurements (time zero, (Tz), control growth, (C), and test growth in the presence of drug at the five concentration levels (Ti)), the percentage growth was calculated at each of the compound concentrations levels. Percentage growth inhibition was calculated as:

[(Ti−Tz)/(C−Tz)]×100 for concentrations for which Ti>/=Tz

[(Ti−Tz)/Tz]×100 for concentrations for which Ti<Tz.

Three dose response parameters were calculated for each compound. Growth inhibition of 50% (GI50) was calculated from [(Ti−Tz)/(C−Tz)]×100=50, which is the compound concentration resulting in a 50% reduction in the net protein increase (as measured by SRB staining) in control cells during the compound incubation. The compound concentration resulting in total growth inhibition (TGI) was calculated from Ti=Tz. The LC50 (concentration of compound resulting in a 50% reduction in the measured protein at the end of the compound treatment as compared to that at the beginning) indicating a net loss of cells following treatment was calculated from [(Ti−Tz)/Tz]×100=−50. Values were calculated for each of these three parameters if the level of activity was reached; however, if the effect was not reached or was exceeded, the value for that parameter was expressed as greater or less than the maximum or minimum concentration tested.

Compounds exhibiting <70% mean percent growth (>30% mean growth inhibition) are scored as positive for anti-tumor activity and seven compounds met this criteria; Compound B, Compound C, Compound A, Compound D, Compound E, Compound F, and Compound G (Table 2). Five of these compounds (Compound C, Compound A, Compound E, Compound F, and Compound G) showed >50% mean growth inhibition and 2 compounds (Compound A and Compound E) showed >90% mean growth inhibition.

TABLE 2

| | | NCI |
|---|---|---|
| | | Screen |
| Com- | | Mean % |
| pound | Structure | Growth |
| B | 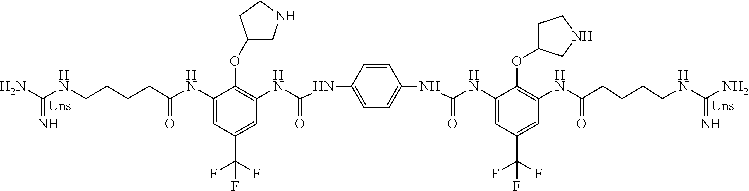 | 56.58 |
| C | 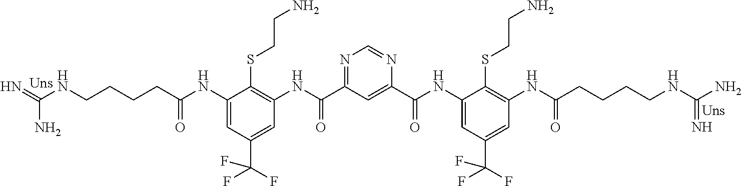 | 43.48 |
| A | 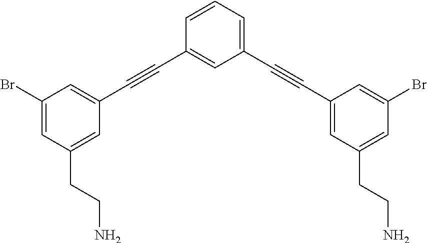 | 6.59 |
| D | 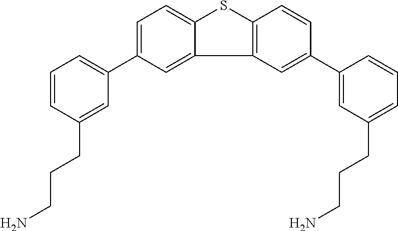 | 67.91 |
| E | 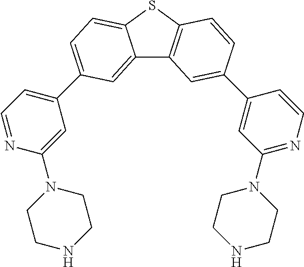 | -42.55 |
| F | 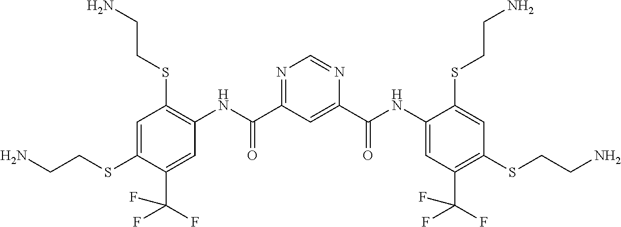 | 41.28 |
| G | 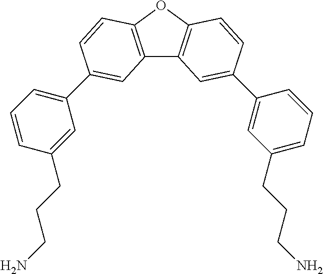 | 49.56 |

TABLE 3
List of tumor cell lines

| Panel Name | Cell Name |
|---|---|
| 1. Leukemia | CCRF-CEM |
| 2. Leukemia | HL-60(TB) |
| 3. Leukemia | K-562 |
| 4. Leukemia | MOLT-4 |
| 5. Leukemia | RPMI-8226 |
| 6. Leukemia | SR |
| 7. Non-Small Cell Lung Cancer | A549/ATCC |
| 8. Non-Small Cell Lung Cancer | EKVX |
| 9. Non-Small Cell Lung Cancer | HOP-62 |
| 10. Non-Small Cell Lung Cancer | HOP-92 |
| 11. Non-Small Cell Lung Cancer | NCI-H226 |
| 12. Non-Small Cell Lung Cancer | NCI-H23 |
| 13. Non-Small Cell Lung Cancer | NCI-H322M |
| 14. Non-Small Cell Lung Cancer | NCI-H460 |
| 15. Non-Small Cell Lung Cancer | NCI-H522 |
| 16. Colon Cancer | COLO 205 |
| 17. Colon Cancer | HCC-2998 |
| 18. Colon Cancer | HCT-116 |
| 19. Colon Cancer | HCT-15 |
| 20. Colon Cancer | HT29 |
| 21. Colon Cancer | KM12 |
| 22. Colon Cancer | SW-620 |
| 23. CNS Cancer | SF-268 |
| 24. CNS Cancer | SF-295 |
| 25. CNS Cancer | SF-539 |
| 26. CNS Cancer | SNB-19 |
| 27. CNS Cancer | SNB-75 |
| 28. CNS Cancer | U251 |
| 29. Melanoma | LOX IMVI |
| 30. Melanoma | MALME-3M |
| 31. Melanoma | MDA-MB-435 |
| 32. Melanoma | SK-MEL-2 |
| 33. Melanoma | SK-MEL-28 |
| 34. Melanoma | SK-MEL-5 |
| 35. Melanoma | UACC-257 |
| 36. Melanoma | UACC-62 |
| 37. Ovarian Cancer | IGROV1 |
| 38. Ovarian Cancer | OVCAR-3 |
| 39. Ovarian Cancer | OVCAR-4 |
| 40. Ovarian Cancer | OVCAR-5 |
| 41. Ovarian Cancer | OVCAR-8 |
| 42. Ovarian Cancer | NCI/ADR-RES |
| 43. Ovarian Cancer | SK-OV-3 |
| 44. Renal Cancer | 786-0 |
| 45. Renal Cancer | A498 |
| 46. Renal Cancer | ACHN |
| 47. Renal Cancer | CAKI-1 |
| 48. Renal Cancer | RXF 393 |
| 49. Renal Cancer | SN12C |
| 50. Renal Cancer | TK-10 |
| 51. Renal Cancer | UO-31 |
| 52. Prostate Cancer | PC-3 |
| 53. Prostate Cancer | DU-145 |
| 54. Breast Cancer | MCF7 |
| 55. Breast Cancer | MDA-MB-31/ATCC |
| 56. Breast Cancer | HS 578T |
| 57. Breast Cancer | BT-549 |
| 58. Breast Cancer | T-47D |
| 59. Breast Cancer | MDA-MB-468 |

TABLE 4

| Compound | Conc. | Cell Type | Cell Name | Percent Growth |
|---|---|---|---|---|
| B | 10 μM | Leukemia | CCRF-CEM | 60.56704867 |
| B | 10 μM | Leukemia | HL-60(TB) | 58.59069586 |
| B | 10 μM | Leukemia | K-562 | 38.56127545 |
| B | 10 μM | Leukemia | MOLT-4 | 63.33176397 |
| B | 10 μM | Leukemia | RPMI-8226 | 49.97221193 |
| B | 10 μM | Leukemia | SR | 77.30857024 |
| B | 10 μM | Non-Small Cell Lung Cancer | A549/ATCC | 49.77802442 |
| B | 10 μM | Non-Small Cell Lung Cancer | EKVX | 61.92472898 |
| B | 10 μM | Non-Small Cell Lung Cancer | HOP-62 | 23.46874008 |
| B | 10 μM | Non-Small Cell Lung Cancer | HOP-92 | 81.50735294 |
| B | 10 μM | Non-Small Cell Lung Cancer | NCI-H226 | 51.21121394 |
| B | 10 μM | Non-Small Cell Lung Cancer | NCI-H23 | 77.57145041 |
| B | 10 μM | Non-Small Cell Lung Cancer | NCI-H322M | 80.76211046 |
| B | 10 μM | Non-Small Cell Lung Cancer | NCI-H460 | 50.72209759 |
| B | 10 μM | Non-Small Cell Lung Cancer | NCI-H522 | −11.8697479 |
| B | 10 μM | Colon Cancer | COLO 205 | 55.40540541 |
| B | 10 μM | Colon Cancer | HCC-2998 | 75.81883623 |
| B | 10 μM | Colon Cancer | HCT-116 | 45.09772509 |
| B | 10 μM | Colon Cancer | HCT-15 | 61.38850362 |
| B | 10 μM | Colon Cancer | HT29 | 59.66371681 |
| B | 10 μM | Colon Cancer | KM12 | 24.15659617 |
| B | 10 μM | Colon Cancer | SW-620 | 69.71194281 |
| B | 10 μM | CNS Cancer | SF-268 | 49.93127148 |
| B | 10 μM | CNS Cancer | SF-295 | 38.86260595 |
| B | 10 μM | CNS Cancer | SF-539 | 30.8 |
| B | 10 μM | CNS Cancer | SNB-19 | 49.15761472 |
| B | 10 μM | CNS Cancer | SNB-75 | 19.47907391 |
| B | 10 μM | CNS Cancer | U251 | 37.7108716 |
| B | 10 μM | Melanoma | LOX IMVI | 59.56966239 |
| B | 10 μM | Melanoma | MALME-3M | 83.13343328 |
| B | 10 μM | Melanoma | MDA-MB-435 | 48.11938623 |
| B | 10 μM | Melanoma | SK-MEL-2 | 60.97542703 |
| B | 10 μM | Melanoma | SK-MEL-28 | 68.28769279 |
| B | 10 μM | Melanoma | SK-MEL-5 | 50.91119271 |
| B | 10 μM | Melanoma | UACC-257 | 75.88824289 |
| B | 10 μM | Melanoma | UACC-62 | 75.90546347 |
| B | 10 μM | Ovarian Cancer | IGROV1 | 82.40082889 |
| B | 10 μM | Ovarian Cancer | OVCAR-3 | 45.05562423 |
| B | 10 μM | Ovarian Cancer | OVCAR-4 | 23.17518248 |
| B | 10 μM | Ovarian Cancer | OVCAR-5 | 77.33056708 |
| B | 10 μM | Ovarian Cancer | OVCAR-8 | 66.5936786 |
| B | 10 μM | Ovarian Cancer | NCI/ADR-RES | 87.71175397 |
| B | 10 μM | Ovarian Cancer | SK-OV-3 | 59.29690103 |
| B | 10 μM | Renal Cancer | 786-0 | 58.60527957 |
| B | 10 μM | Renal Cancer | A498 | 66.4491654 |
| B | 10 μM | Renal Cancer | ACHN | 62.44874715 |
| B | 10 μM | Renal Cancer | CAKI-1 | 56.4196327 |
| B | 10 μM | Renal Cancer | RXF 393 | 87.8436019 |
| B | 10 μM | Renal Cancer | SN12C | 64.79662318 |
| B | 10 μM | Renal Cancer | TK-10 | 60.44912164 |
| B | 10 μM | Renal Cancer | UO-31 | 45.66748316 |
| B | 10 μM | Prostate Cancer | PC-3 | 59.59075289 |
| B | 10 μM | Prostate Cancer | DU-145 | 70.36192214 |
| B | 10 μM | Breast Cancer | MCF7 | 44.40500338 |
| B | 10 μM | Breast Cancer | MDA-MB-231/ATCC | 73.01694004 |
| B | 10 μM | Breast Cancer | BT-549 | 89.60646521 |
| B | 10 μM | Breast Cancer | T-47D | 3.785792952 |
| B | 10 μM | Breast Cancer | MDA-MB-468 | 43.01872075 |
| C | 10 μM | Leukemia | CCRF-CEM | 24.46205292 |
| C | 10 μM | Leukemia | HL-60(TB) | 30.10763209 |
| C | 10 μM | Leukemia | K-562 | 4.995242626 |
| C | 10 μM | Leukemia | MOLT-4 | 45.62090459 |
| C | 10 μM | Leukemia | SR | 28.11585111 |
| C | 10 μM | Non-Small Cell Lung Cancer | A549/ATCC | 37.25989523 |
| C | 10 μM | Non-Small Cell Lung Cancer | EKVX | 59.81830123 |
| C | 10 μM | Non-Small Cell Lung Cancer | HOP-62 | 37.78474744 |
| C | 10 μM | Non-Small Cell Lung Cancer | HOP-92 | 93.9884114 |
| C | 10 μM | Non-Small Cell Lung Cancer | NCI-H226 | 63.81762409 |
| C | 10 μM | Non-Small Cell Lung Cancer | NCI-H23 | 80.24168719 |
| C | 10 μM | Non-Small Cell Lung Cancer | NCI-H322M | 83.13478557 |
| C | 10 μM | Non-Small Cell Lung Cancer | NCI-H460 | 28.71806232 |
| C | 10 μM | Non-Small Cell Lung Cancer | NCI-H522 | −26.8697479 |
| C | 10 μM | Colon Cancer | COLO 205 | −73.19852941 |
| C | 10 μM | Colon Cancer | HCC-2998 | 54.19813132 |
| C | 10 μM | Colon Cancer | HCT-116 | 37.88448394 |
| C | 10 μM | Colon Cancer | HCT-15 | 33.27456692 |
| C | 10 μM | Colon Cancer | HT29 | 17.03476483 |
| C | 10 μM | Colon Cancer | KM12 | −39.48353293 |
| C | 10 μM | Colon Cancer | SW-620 | 34.38645676 |
| C | 10 μM | CNS Cancer | SF-268 | 53.23563892 |
| C | 10 μM | CNS Cancer | SF-295 | 39.81144934 |
| C | 10 μM | CNS Cancer | SF-539 | 70.30023548 |
| C | 10 μM | CNS Cancer | SNB-19 | 47.24922931 |
| C | 10 μM | CNS Cancer | SNB-75 | 22.46929267 |
| C | 10 μM | CNS Cancer | U251 | 31.27525253 |
| C | 10 μM | Melanoma | LOX IMVI | 50.64316954 |
| C | 10 μM | Melanoma | MALME-3M | 63.61909929 |
| C | 10 μM | Melanoma | MDA-MB-435 | 35.56282204 |
| C | 10 μM | Melanoma | SK-MEL-2 | 54.29003502 |
| C | 10 μM | Melanoma | SK-MEL-28 | 61.38908328 |
| C | 10 μM | Melanoma | SK-MEL-5 | 34.55048213 |
| C | 10 μM | Melanoma | UACC-257 | 61.308507 |
| C | 10 μM | Melanoma | UACC-62 | 55.9264585 |
| C | 10 μM | Ovarian Cancer | IGROV1 | 51.71428571 |
| C | 10 μM | Ovarian Cancer | OVCAR-3 | 37.56399317 |
| C | 10 μM | Ovarian Cancer | OVCAR-4 | 38.78800676 |
| C | 10 μM | Ovarian Cancer | OVCAR-5 | 75.78168621 |
| C | 10 μM | Ovarian Cancer | OVCAR-8 | 39.11131899 |
| C | 10 μM | Ovarian Cancer | NCI/ADR-RES | 55.27247956 |
| C | 10 μM | Ovarian Cancer | SK-OV-3 | 82.66460905 |
| C | 10 μM | Renal Cancer | A498 | 69.49638118 |
| C | 10 μM | Renal Cancer | ACHN | 53.07987365 |
| C | 10 μM | Renal Cancer | CAKI-1 | 47.81213536 |
| C | 10 μM | Renal Cancer | RXF 393 | 78.0583874 |
| C | 10 μM | Renal Cancer | SN12C | 65.97278471 |
| C | 10 μM | Renal Cancer | TK-10 | 38.30068819 |
| C | 10 μM | Renal Cancer | UO-31 | 73.63693665 |
| C | 10 μM | Prostate Cancer | PC-3 | 77.68333333 |
| C | 10 μM | Prostate Cancer | DU-145 | 62.89416172 |
| C | 10 μM | Breast Cancer | MCF7 | 10.26768642 |
| C | 10 μM | Breast Cancer | MDA-MB-231/ATCC | 81.51820065 |
| C | 10 μM | Breast Cancer | BT-549 | 96.66713444 |
| C | 10 μM | Breast Cancer | T-47D | 14.59387087 |
| C | 10 μM | Breast Cancer | MDA-MB-468 | −53.05365297 |
| A | 10 μM | Leukemia | CCRF-CEM | −12.42895036 |
| A | 10 μM | Leukemia | HL-60(TB) | 6.879952902 |
| A | 10 μM | Leukemia | K-562 | −45.66544567 |
| A | 10 μM | Leukemia | MOLT-4 | −43.64601262 |
| A | 10 μM | Leukemia | RPMI-8226 | 9.49729175 |
| A | 10 μM | Leukemia | SR | −43.86759582 |
| A | 10 μM | Non-Small Cell Lung Cancer | A549/ATCC | −15.97082495 |
| A | 10 μM | Non-Small Cell Lung Cancer | EKVX | 62.4761214 |
| A | 10 μM | Non-Small Cell Lung Cancer | HOP-62 | 21.994747 |
| A | 10 μM | Non-Small Cell Lung Cancer | HOP-92 | 73.03084054 |
| A | 10 μM | Non-Small Cell Lung Cancer | NCI-H226 | 108.9198494 |
| A | 10 μM | Non-Small Cell Lung Cancer | NCI-H23 | 94.81453277 |
| A | 10 μM | Non-Small Cell Lung Cancer | NCI-H322M | 53.00421156 |
| A | 10 μM | Non-Small Cell Lung Cancer | NCI-H460 | −49.10714286 |
| A | 10 μM | Non-Small Cell Lung Cancer | NCI-H522 | −78.21128451 |
| A | 10 μM | Colon Cancer | COLO 205 | 1.202942583 |
| A | 10 μM | Colon Cancer | HCC-2998 | 54.09358263 |
| A | 10 μM | Colon Cancer | HCT-116 | −45.86466165 |
| A | 10 μM | Colon Cancer | HCT-15 | 11.61260723 |
| A | 10 μM | Colon Cancer | HT29 | −89.30735931 |
| A | 10 μM | Colon Cancer | KM12 | −87.5320787 |
| A | 10 μM | Colon Cancer | SW-620 | −73.57910906 |
| A | 10 μM | CNS Cancer | SF-268 | 61.54884919 |
| A | 10 μM | CNS Cancer | SF-295 | 3.169591962 |
| A | 10 μM | CNS Cancer | SF-539 | 55.6-43247451 |
| A | 10 μM | CNS Cancer | SNB-19 | 27.06409377 |
| A | 10 μM | CNS Cancer | SNB-75 | −2.086919105 |
| A | 10 μM | CNS Cancer | U251 | −64.64829587 |
| A | 10 μM | Melanoma | LOX IMVI | −76.90622261 |
| A | 10 μM | Melanoma | MALME-3M | 106.7649941 |
| A | 10 μM | Melanoma | MDA-MB-435 | −16.18629908 |
| A | 10 μM | Melanoma | SK-MEL-2 | 3.514550579 |
| A | 10 μM | Melanoma | SK-MEL-28 | 79.76282372 |
| A | 10 μM | Melanoma | SK-MEL-5 | 80.49932759 |
| A | 10 μM | Melanoma | UACC-257 | 68.25206834 |
| A | 10 μM | Melanoma | UACC-62 | 26.02653411 |
| A | 10 μM | Ovarian Cancer | IGROV1 | −4.831932773 |
| A | 10 μM | Ovarian Cancer | OVCAR-3 | −35.66189312 |
| A | 10 μM | Ovarian Cancer | OVCAR-4 | −28.49597586 |
| A | 10 μM | Ovarian Cancer | OVCAR-5 | 60.76782917 |
| A | 10 μM | Ovarian Cancer | OVCAR-8 | −14.53561016 |
| A | 10 μM | Ovarian Cancer | NCI/ADR-RES | 23.97458346 |
| A | 10 μM | Ovarian Cancer | SK-OV-3 | 94.37345275 |
| A | 10 μM | Renal Cancer | 786-0 | −26.77949228 |
| A | 10 μM | Renal Cancer | A498 | 84.00588761 |
| A | 10 μM | Renal Cancer | ACHN | −46.90572585 |
| A | 10 μM | Renal Cancer | CAKI-1 | 70.65983701 |
| A | 10 μM | Renal Cancer | RXF 393 | −26.13019892 |
| A | 10 μM | Renal Cancer | SN12C | 22.79578332 |
| A | 10 μM | Renal Cancer | TK-10 | 8.048093285 |
| A | 10 μM | Renal Cancer | UO-31 | 37.26004745 |
| A | 10 μM | Prostate Cancer | PC-3 | 45.2550021 |
| A | 10 μM | Prostate Cancer | DU-145 | −83.92409595 |
| A | 10 μM | Breast Cancer | MCF7 | −89.84126984 |
| A | 10 μM | Breast Cancer | MDA-MB-231/ATCC | 4.310420354 |
| A | 10 μM | Breast Cancer | BT-549 | 101.9508945 |
| A | 10 μM | Breast Cancer | T-47D | 31.81838363 |
| A | 10 μM | Breast Cancer | MDA-MB-468 | −61.49706458 |
| D | 10 μM | Leukemia | CCRF-CEM | 5.119809495 |
| D | 10 μM | Leukemia | HL-60(TB) | 68.33463643 |
| D | 10 μM | Leukemia | K-562 | 12.98501729 |
| D | 10 μM | Leukemia | MOLT-4 | 2.071563089 |
| D | 10 μM | Leukemia | RPMI-8226 | 62.06002223 |
| D | 10 μM | Leukemia | SR | 0 |
| D | 10 μM | Non-Small Cell Lung Cancer | A549/ATCC | 72.71285873 |
| D | 10 μM | Non-Small Cell Lung Cancer | EKVX | 98.17958683 |
| D | 10 μM | Non-Small Cell Lung Cancer | HOP-62 | 90.63789273 |
| D | 10 μM | Non-Small Cell Lung Cancer | HOP-92 | 78.26470588 |
| D | 10 μM | Non-Small Cell Lung Cancer | NCI-H226 | 110.0707676 |
| D | 10 μM | Non-Small Cell Lung Cancer | NCI-H23 | 100.5960569 |
| D | 10 μM | Non-Small Cell Lung Cancer | NCI-H322M | 107.0040111 |
| D | 10 μM | Non-Small Cell Lung Cancer | NCI-H460 | 69.5737521 |
| D | 10 μM | Non-Small Cell Lung Cancer | NCI-H522 | 10.74540174 |
| D | 10 μM | Colon Cancer | COLO 205 | 31.52204836 |
| D | 10 μM | Colon Cancer | HCC-2998 | 93.26934613 |
| D | 10 μM | Colon Cancer | HCT-116 | 0.5126562 |
| D | 10 μM | Colon Cancer | HCT-15 | 88.14917553 |
| D | 10 μM | Colon Cancer | HT29 | 21.30973451 |
| D | 10 μM | Colon Cancer | KM12 | 42.09466264 |
| D | 10 μM | Colon Cancer | SW-620 | 15.47518923 |
| D | 10 μM | CNS Cancer | SF-268 | 73.40206186 |
| D | 10 μM | CNS Cancer | SF-295 | 87.16735659 |
| D | 10 μM | CNS Cancer | SF-539 | 98.34418605 |
| D | 10 μM | CNS Cancer | SNB-19 | 79.89359344 |
| D | 10 μM | CNS Cancer | SNB-75 | 71.14870882 |
| D | 10 μM | CNS Cancer | U251 | 59.70009372 |
| D | 10 μM | Melanoma | LOX IMVI | 26.77127913 |
| D | 10 μM | Melanoma | MALME-3M | 134.1329335 |
| D | 10 μM | Melanoma | MDA-MB-435 | 81.13772455 |
| D | 10 μM | Melanoma | SK-MEL-2 | 81.00089901 |
| D | 10 μM | Melanoma | SK-MEL-28 | 99.21309411 |
| D | 10 μM | Melanoma | SK-MEL-5 | 100.8023936 |
| D | 10 μM | Melanoma | UACC-257 | 100 |
| D | 10 μM | Melanoma | UACC-62 | 88.43871496 |
| D | 10 μM | Ovarian Cancer | IGROV1 | 80.28419183 |
| D | 10 μM | Ovarian Cancer | OVCAR-3 | 62.71116605 |
| D | 10 μM | Ovarian Cancer | OVCAR-4 | 59.8540146 |
| D | 10 μM | Ovarian Cancer | OVCAR-5 | 96.92946058 |
| D | 10 μM | Ovarian Cancer | OVCAR-8 | 83.89185072 |
| D | 10 μM | Ovarian Cancer | NCI/ADR-RES | 95.38702111 |
| D | 10 μM | Ovarian Cancer | SK-OV-3 | 103.098967 |
| D | 10 μM | Renal Cancer | 786-0 | 79.48451465 |
| D | 10 μM | Renal Cancer | A498 | 81.63884674 |
| D | 10 μM | Renal Cancer | ACHN | 68.97494305 |
| D | 10 μM | Renal Cancer | CAKI-1 | 82.82138794 |
| D | 10 μM | Renal Cancer | RXF 393 | 81.70616114 |
| D | 10 μM | Renal Cancer | SN12C | 84.45126631 |
| D | 10 μM | Renal Cancer | TK-10 | 86.47663242 |
| D | 10 μM | Renal Cancer | UO-31 | 85.60930802 |
| D | 10 μM | Prostate Cancer | PC-3 | 73.60199938 |
| D | 10 μM | Prostate Cancer | DU-145 | 55.35279805 |
| D | 10 μM | Breast Cancer | MCF7 | −59.62962963 |
| D | 10 μM | Breast Cancer | MDA-MB-231/ATCC | 60.17746706 |
| D | 10 μM | Breast Cancer | BT-549 | 105.7765285 |
| D | 10 μM | Breast Cancer | T-47D | 90.74889868 |
| D | 10 μM | Breast Cancer | MDA-MB-468 | 17.62870515 |
| E | 10 μM | Leukemia | CCRF-CEM | −64.02519894 |
| E | 10 μM | Leukemia | HL-60(TB) | −24.59288299 |
| E | 10 μM | Leukemia | K-562 | −70.88675214 |
| E | 10 μM | Leukemia | MOLT-4 | −75.27610442 |
| E | 10 μM | Leukemia | SR | −60.70121951 |
| E | 10 μM | Non-Small Cell Lung Cancer | A549/ATCC | −77.24471831 |
| E | 10 μM | Non-Small Cell Lung Cancer | EKVX | 22.55910683 |
| E | 10 μM | Non-Small Cell Lung Cancer | HOP-62 | −80.18149883 |
| E | 10 μM | Non-Small Cell Lung Cancer | HOP-92 | 32.76195075 |
| E | 10 μM | Non-Small Cell Lung Cancer | NCI-H226 | 39.89124373 |
| E | 10 μM | Non-Small Cell Lung Cancer | NCI-H23 | 52.19365764 |
| E | 10 μM | Non-Small Cell Lung Cancer | NCI-H322M | −53.02230047 |
| E | 10 μM | Non-Small Cell Lung Cancer | NCI-H460 | −78.46467391 |
| E | 10 μM | Non-Small Cell Lung Cancer | NCI-H522 | −93.92857143 |
| E | 10 μM | Colon Cancer | COLO 205 | −59.52205882 |
| E | 10 μM | Colon Cancer | HCC-2998 | −9.442771084 |
| E | 10 μM | Colon Cancer | HCT-116 | −48.51973684 |
| E | 10 μM | Colon Cancer | HCT-15 | −54.01291513 |
| E | 10 μM | Colon Cancer | HT29 | −87.5 |
| E | 10 μM | Colon Cancer | KM12 | −87.83682635 |
| E | 10 μM | Colon Cancer | SW-620 | −87.2983871 |
| E | 10 μM | CNS Cancer | SF-268 | −37.04514825 |
| E | 10 μM | CNS Cancer | SF-295 | −50.64713896 |
| E | 10 μM | CNS Cancer | SF-539 | 14.25627943 |
| E | 10 μM | CNS Cancer | SNB-19 | −43.9699793 |
| E | 10 μM | CNS Cancer | SNB-75 | −94.78539157 |
| E | 10 μM | CNS Cancer | U251 | −85.72335025 |
| E | 10 μM | Melanoma | LOX IMVI | −80.29141104 |
| E | 10 μM | Melanoma | MALME-3M | −4.678571429 |
| E | 10 μM | Melanoma | MDA-MB-435 | −84.02777778 |
| E | 10 μM | Melanoma | SK-MEL-2 | −73.4777937 |
| E | 10 μM | Melanoma | SK-MEL-28 | −51.91498316 |
| E | 10 μM | Melanoma | SK-MEL-5 | 63.84713556 |
| E | 10 μM | Melanoma | UACC-257 | −70.36713287 |
| E | 10 μM | Melanoma | UACC-62 | −41.06675393 |
| E | 10 μM | Ovarian Cancer | IGROV1 | −43.56617647 |
| E | 10 μM | Ovarian Cancer | OVCAR-3 | −94.46821516 |
| E | 10 μM | Ovarian Cancer | OVCAR-4 | −87.74207746 |
| E | 10 μM | Ovarian Cancer | OVCAR-5 | −46.6609589 |
| E | 10 μM | Ovarian Cancer | OVCAR-8 | −61.04227405 |
| E | 10 μM | Ovarian Cancer | NCI/ADR-RES | 3.119891008 |
| E | 10 μM | Ovarian Cancer | SK-OV-3 | 51.66323731 |
| E | 10 μM | Renal Cancer | A498 | 75.10554885 |
| E | 10 μM | Renal Cancer | ACHN | −69.18016194 |
| E | 10 μM | Renal Cancer | CAKI-1 | −13.75584112 |
| E | 10 μM | Renal Cancer | RXF 393 | −70.64873418 |
| E | 10 μM | Renal Cancer | SN12C | −16.99260042 |
| E | 10 μM | Renal Cancer | TK-10 | −52.46010638 |
| E | 10 μM | Renal Cancer | UO-31 | −41.12394958 |
| E | 10 μM | Prostate Cancer | PC-3 | −85.1070529 |
| E | 10 μM | Prostate Cancer | DU-145 | −97.96365915 |
| E | 10 μM | Breast Cancer | MCF7 | −89.84567901 |
| E | 10 μM | Breast Cancer | MDA-MB-231/ATCC | −41.25647668 |
| E | 10 μM | Breast Cancer | BT-549 | 98.88436711 |
| E | 10 μM | Breast Cancer | T-47D | −12.2723824 |
| E | 10 μM | Breast Cancer | MDA-MB-468 | −82.27739726 |
| F | 10 μM | Leukemia | CCRF-CEM | 99.09391406 |
| F | 10 μM | Leukemia | HL-60(TB) | 82.5679772 |
| F | 10 μM | Leukemia | K-562 | 96.34397606 |
| F | 10 μM | Leukemia | MOLT-4 | 82.84538507 |
| F | 10 μM | Leukemia | SR | 51.7002547 |
| F | 10 μM | Non-Small Cell Lung Cancer | A549/ATCC | 15.80792281 |
| F | 10 μM | Non-Small Cell Lung Cancer | EKVX | 75.72482416 |
| F | 10 μM | Non-Small Cell Lung Cancer | HOP-62 | 29.35413958 |
| F | 10 μM | Non-Small Cell Lung Cancer | HOP-92 | −24.05080214 |
| F | 10 μM | Non-Small Cell Lung Cancer | NCI-H226 | 66.8476212 |
| F | 10 μM | Non-Small Cell Lung Cancer | NCI-H23 | 60.86059885 |
| F | 10 μM | Non-Small Cell Lung Cancer | NCI-H322M | 55.47536309 |
| F | 10 μM | Non-Small Cell Lung Cancer | NCI-H460 | 47.7345486 |
| F | 10 μM | Non-Small Cell Lung Cancer | NCI-H522 | −2.5 |
| F | 10 μM | Colon Cancer | COLO 205 | 126.0155836 |
| F | 10 μM | Colon Cancer | HCC-2998 | 31.92868083 |
| F | 10 μM | Colon Cancer | HCT-116 | 32.40675855 |
| F | 10 μM | Colon Cancer | HCT-15 | 62.31243024 |
| F | 10 μM | Colon Cancer | HT29 | 56.33772168 |
| F | 10 μM | Colon Cancer | KM12 | 7.629710856 |
| F | 10 μM | Colon Cancer | SW-620 | 18.56788414 |
| F | 10 μM | CNS Cancer | SF-268 | 32.42375602 |
| F | 10 μM | CNS Cancer | SF-295 | 57.6580429 |
| F | 10 μM | CNS Cancer | SF-539 | 65.72664637 |
| F | 10 μM | CNS Cancer | SNB-19 | 49.53721665 |
| F | 10 μM | CNS Cancer | SNB-75 | −20.2371988 |
| F | 10 μM | CNS Cancer | U251 | 23.67199945 |
| F | 10 μM | Melanoma | LOX IMVI | 6.660405981 |
| F | 10 μM | Melanoma | MALME-3M | 80.14837203 |
| F | 10 μM | Melanoma | MDA-MB-435 | 70.6223935 |
| F | 10 μM | Melanoma | SK-MEL-2 | 46.77454721 |
| F | 10 μM | Melanoma | SK-MEL-28 | 83.93614143 |
| F | 10 μM | Melanoma | SK-MEL-5 | 62.30274693 |
| F | 10 μM | Melanoma | UACC-257 | 76.45930392 |
| F | 10 μM | Melanoma | UACC-62 | 51.22618728 |
| F | 10 μM | Ovarian Cancer | IGROV1 | 4.69990643 |
| F | 10 μM | Ovarian Cancer | OVCAR-3 | −15.49511002 |
| F | 10 μM | Ovarian Cancer | OVCAR-4 | −1.474471831 |
| F | 10 μM | Ovarian Cancer | OVCAR-5 | 59.37378603 |
| F | 10 μM | Ovarian Cancer | OVCAR-8 | 14.3368814 |
| F | 10 μM | Ovarian Cancer | NCI/ADR-RES | 54.75512331 |
| F | 10 μM | Ovarian Cancer | SK-OV-3 | 15.33182844 |
| F | 10 μM | Renal Cancer | 786-0 | 50.08773178 |
| F | 10 μM | Renal Cancer | A498 | 69.60307298 |
| F | 10 μM | Renal Cancer | ACHN | 20.01875586 |
| F | 10 μM | Renal Cancer | CAKI-1 | 31.42207895 |
| F | 10 μM | Renal Cancer | RXF 393 | 47.68196833 |
| F | 10 μM | Renal Cancer | SN12C | 57.45582742 |
| F | 10 μM | Renal Cancer | TK-10 | 21.47681845 |
| F | 10 μM | Renal Cancer | UO-31 | 48.73731222 |
| F | 10 μM | Prostate Cancer | PC-3 | 46.60524197 |
| F | 10 μM | Prostate Cancer | DU-145 | 51.96752996 |
| F | 10 μM | Breast Cancer | MCF7 | −59.9691358 |
| F | 10 μM | Breast Cancer | MDA-MB-231/ATCC | 18.8735423 |
| F | 10 μM | Breast Cancer | BT-549 | 101.7909055 |
| F | 10 μM | Breast Cancer | T-47D | 34.10789063 |
| F | 10 μM | Breast Cancer | MDA-MB-468 | −78.28196347 |
| G | 10 μM | Leukemia | CCRF-CEM | 9.164625391 |
| G | 10 μM | Leukemia | HL-60(TB) | 46.37242457 |
| G | 10 μM | Leukemia | K-562 | 2.899311452 |
| G | 10 μM | Leukemia | MOLT-4 | −6.40060241 |
| G | 10 μM | Leukemia | RPMI-8226 | 65.28202886 |
| G | 10 μM | Leukemia | SR | −19.60365854 |
| G | 10 μM | Non-Small Cell Lung Cancer | EKVX | 81.68765447 |
| G | 10 μM | Non-Small Cell Lung Cancer | HOP-62 | 72.69260107 |
| G | 10 μM | Non-Small Cell Lung Cancer | HOP-92 | 61.60445166 |
| G | 10 μM | Non-Small Cell Lung Cancer | NCI-H226 | 104.4474949 |
| G | 10 μM | Non-Small Cell Lung Cancer | NCI-H23 | 99.37669154 |
| G | 10 μM | Non-Small Cell Lung Cancer | NCI-H322M | 108.3441982 |
| G | 10 μM | Non-Small Cell Lung Cancer | NCI-H460 | 23.90914953 |
| G | 10 μM | Non-Small Cell Lung Cancer | NCI-H522 | 69.45052654 |
| G | 10 μM | Colon Cancer | COLO 205 | 26.36610257 |
| G | 10 μM | Colon Cancer | HCC-2998 | 81.22292451 |
| G | 10 μM | Colon Cancer | HCT-116 | −37.99342105 |
| G | 10 μM | Colon Cancer | HCT-15 | 49.70960678 |
| G | 10 μM | Colon Cancer | HT29 | −51.13636364 |
| G | 10 μM | Colon Cancer | KM12 | 14.0126598 |
| G | 10 μM | Colon Cancer | SW-620 | −7.997311828 |
| G | 10 μM | CNS Cancer | SF-268 | 75.18300732 |
| G | 10 μM | CNS Cancer | SF-295 | 65.39843847 |
| G | 10 μM | CNS Cancer | SF-539 | 86.72985782 |
| G | 10 μM | CNS Cancer | SNB-19 | 88.57278895 |
| G | 10 μM | CNS Cancer | SNB-75 | 66.47484685 |
| G | 10 μM | Melanoma | LOX IMVI | −75.07668712 |
| G | 10 μM | Melanoma | MALME-3M | 105.0284346 |
| G | 10 μM | Melanoma | MDA-MB-435 | 62.07352552 |
| G | 10 μM | Melanoma | SK-MEL-2 | 72.07089695 |
| G | 10 μM | Melanoma | SK-MEL-28 | 102.1197194 |
| G | 10 μM | Melanoma | SK-MEL-5 | 89.14768695 |
| G | 10 μM | Melanoma | UACC-257 | 44.84288355 |
| G | 10 μM | Melanoma | UACC-62 | 72.9182058 |
| G | 10 μM | Ovarian Cancer | IGROV1 | 42.20102455 |
| G | 10 μM | Ovarian Cancer | OVCAR-3 | 18.53691578 |
| G | 10 μM | Ovarian Cancer | OVCAR-4 | 54.91064109 |
| G | 10 μM | Ovarian Cancer | OVCAR-5 | 64.4761766 |
| G | 10 μM | Ovarian Cancer | OVCAR-8 | 33.74079733 |
| G | 10 μM | Ovarian Cancer | NCI/ADR-RES | 83.66303324 |
| G | 10 μM | Ovarian Cancer | SK-OV-3 | 94.44724886 |
| G | 10 μM | Renal Cancer | 786-0 | 5.486602482 |
| G | 10 μM | Renal Cancer | A498 | 92.25604996 |
| G | 10 μM | Renal Cancer | ACHN | 36.71216617 |
| G | 10 μM | Renal Cancer | CAKI-1 | 56.68454058 |
| G | 10 μM | Renal Cancer | RXF 393 | 65.04720239 |
| G | 10 μM | Renal Cancer | SN12C | 82.21762318 |
| G | 10 μM | Renal Cancer | TK-10 | 57.58849744 |
| G | 10 μM | Renal Cancer | UO-31 | 84.95418743 |
| G | 10 μM | Prostate Cancer | PC-3 | 54.99433382 |
| G | 10 μM | Prostate Cancer | DU-145 | −23.65288221 |
| G | 10 μM | Breast Cancer | MCF7 | −44.29012346 |
| G | 10 μM | Breast Cancer | MDA-MB-231/ATCC | 37.18840088 |
| G | 10 μM | Breast Cancer | HS 578T | 100.3650745 |
| G | 10 μM | Breast Cancer | BT-549 | 101.5045208 |
| G | 10 μM | Breast Cancer | T-47D | 80.44246629 |
| G | 10 μM | Breast Cancer | MDA-MB-468 | −3.510273973 |

Having now fully described this invention, it will be understood to those of ordinary skill in the art that the same can be performed within a wide and equivalent range of conditions, formulations, and other parameters without affecting the scope of the invention or any embodiment thereof. All documents, e.g., scientific publications, patents, patent applications and patent publications recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A method for treating or reducing cancer, or inhibiting growth of a cancer cell, or inhibiting tumor growth, or reducing spread or metastasis of cancer in an animal in need thereof comprising administering to the animal an effective amount of a compound or pharmaceutically acceptable salt thereof; wherein the compound or pharmaceutically acceptable salt administered is a compound of Formula I: 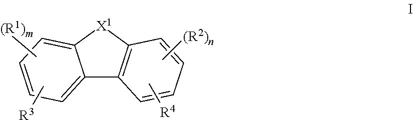
or pharmaceutically acceptable salt thereof, wherein:
X¹ is O, S, S(═O), or S(═O)₂;
R¹ and R² are, independently, halo, C₁₋₄ alkyl, C₁₋₄ alkoxy, CN, C₁₋₄haloalkyl, or C₁₋₄ haloalkoxy;
R³ and R⁴ are, independently, phenyl, pyridinyl, pyrimidinyl, pyrazinyl, or 1,2,3,6-tetrahydropyridin-4-yl, each substituted with R⁵ and optionally substituted with R⁶;
each R⁵ is independently —(CH₂)ₜ₁—NH₂, —(CH₂)ₜ₂—NH—(CH₂)ₜ₃—NH₂, —(CH₂)ₜ₄—NHC(═NH)NH₂, —S—(CH₂)ₜ₅—NH₂, —O—(CH₂)ₜ₆—NH₂, —(CH₂)ₜ₇—NH₂, or NR⁷R⁸;
each R⁶ is independently halo, OH, C₁₋₄ alkyl, C₁₋₄ alkoxy, CN, C₁₋₄haloalkyl, or C₁₋₄haloalkoxy;
R⁷ and R⁸, together with the N atom to which they are attached, form pyrrolidin-1-yl, piperidin-1-yl, morpholin-1-yl, or piperazin-1-yl, each optionally substituted with 1 or 2 C₁₋₄ alkyl;
m is 0 or 1;
n is 0 or 1;
each t1 is independently 2 or 3;
each t2 is independently 1, 2, or 3;
each t3 is independently 2 or 3;
each t4 is independently 2 or 3;
each t5 is independently 2 or 3;
each t6 is independently 2 or 3; and
each t7 is independently 2 or 3;
or the compound or pharmaceutically acceptable salt administered is a compound of Formula II: 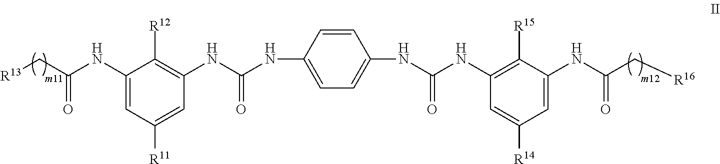
or pharmaceutically acceptable salt thereof, wherein:
R¹¹ and R¹⁴ are, independently, H, Cl, CN, methyl, CH₂F, CHF₂, or CF₃;
R¹² and R¹⁵ are, independently, —S—(CH₂)ₜ₁₁—NH₂, —O—(CH₂)ₜ₁₂—NH₂, —O—R¹⁷, —S—(CH₂)ₜ₁₃—NHC(═NH)NH₂, or —(CH₂)ₜ₁₄—NH₂;
R¹³ and R¹⁶ are, independently, —NH₂, —NH—(CH₂)ₘ₁₃—NH₂, —NHC(═NH)NH₂, or —NH—(CH₂)ₘ₁₄—NHC(═NH)NH₂;
each R¹⁷ is independently pyrrolidinyl, piperidinyl, morpholinyl, or piperazinyl, each optionally substituted with 1 or 2 C₁₋₄ alkyl;
each of t11, t12, t13, and t14 is independently 2 or 3;
each of m11 and m12 is independently 3, 4, or 5;
each of m13 and m14 is independently 1, 2, 3, 4, or 5;
or the compound or pharmaceutically acceptable salt administered is a compound of Formula III: 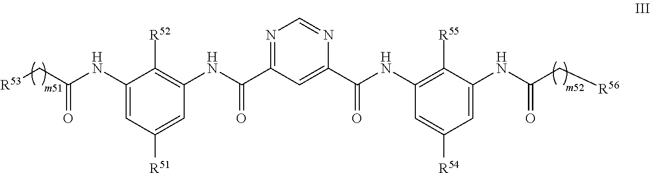
or pharmaceutically acceptable salt thereof, wherein:
R⁵¹ and R⁵⁴ are, independently, H, Cl, CN, methyl, CH₂F, CHF₂, or CF₃;
R⁵² and R⁵⁵ are, independently, —S—(CH₂)ₜ₅₁—NH₂ or —(CH₂)ₜ₅₂—NH₂;
R⁵³ and R⁵⁶ are, independently, —NH₂, —NH—(CH₂)ₘ₅₃—NH₂, —NHC(═NH)NH₂, or —NH—(CH₂)ₘ₅₄—NHC(═NH)NH₂;
each of t51 and t52 is independently 2 or 3;
each of m51 and m52 is independently 3, 4, or 5; and
each of m53 and m54 is independently 1, 2, 3, 4, or 5;
or the compound or pharmaceutically acceptable salt administered is a compound of Formula IV or IVa: 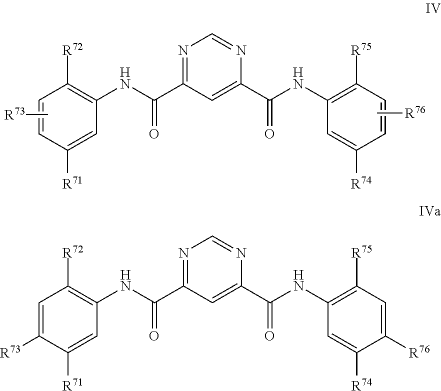
or pharmaceutically acceptable salt thereof, wherein:
R⁷¹ and R⁷⁴ are, independently, H, Cl, CN, methyl, CH₂F, CHF₂, or CF₃;
R⁷² and R⁷⁵ are, independently, —S—(CH₂)ₜ₇₁—NH₂, —(CH₂)ₜ₇₂—NH₂, or —O—(CH₂)ₜ₇₃—NH₂;
R⁷³ and R⁷⁶ are, independently, —S—(CH₂)ₜ₇₄—NH₂, —(CH₂)ₜ₇₅—NH₂, or —O—(CH₂)ₜ₇₆—NH₂;
t71 and t74 are independently 2 or 3;
t72 and t75 are independently 2 or 3; and
t73 and t76 are independently 2 or 3.

2. The method of claim 1 wherein the compound of Formula I or pharmaceutically acceptable salt thereof is a compound Formula Ia: 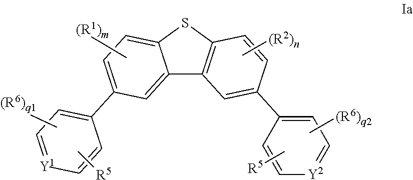
or pharmaceutically acceptable salt thereof, wherein:
R¹ and R² are, independently, Cl, Br, methyl, CH₂F, CHF₂, or CF₃;
each R⁵ is independently —(CH₂)₂—NH₂, —(CH₂)₃—NH₂, —S—(CH₂)₂—NH₂, —O—(CH₂)₃—NH₂, or piperazin-1-yl;
each R⁶ is independently, Cl, Br, methyl, CH₂F, CHF₂, or CF₃;
Y¹ is N or CH;
Y² is N or CH;
m is 0 or 1;
n is 0 or 1;
q1 is 0 or 1; and
q2 is 0 or 1.

3. The method of claim 2 wherein the compound of Formula Ia or pharmaceutically acceptable salt thereof is a compound Formula Ia-1: 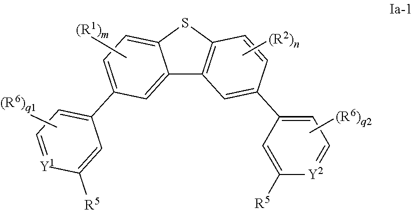
or pharmaceutically acceptable salt thereof.

4. The method of claim 3 wherein the compound of Formula Ia-1 or pharmaceutically acceptable salt thereof is a compound of Formula Ia-1-1: 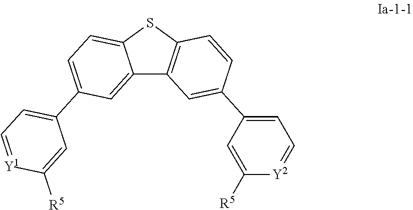
or pharmaceutically acceptable salt thereof.

5. The method of claim 2 wherein the compound of Formula Ia or pharmaceutically acceptable salt thereof is a compound selected from: 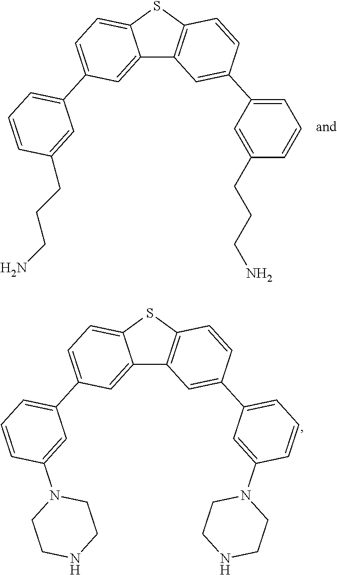
or pharmaceutically acceptable salt thereof.

6. The method of claim 1 wherein the compound of Formula I or pharmaceutically acceptable salt thereof is a compound Formula Ib: 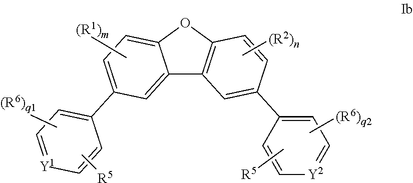
or pharmaceutically acceptable salt thereof, wherein:
R¹ and R² are, independently, Cl, Br, methyl, CH₂F, CHF₂, or CF₃;
each R⁵ is independently —(CH₂)₂—NH₂, —(CH₂)₃—NH₂, —S—(CH₂)₂—NH₂, —O—(CH₂)₃—NH₂, or piperazin-1-yl;
each R⁶ is independently, Cl, Br, methyl, CH₂F, CHF₂, or CF₃;
Y¹ is N or CH;
Y² is N or CH;
m is 0 or 1;
n is 0 or 1;
q1 is 0 or 1; and
q2 is 0 or 1.

7. The method of claim 6 wherein the compound of Formula Ib or pharmaceutically acceptable salt thereof is a compound Formula Ib-1: 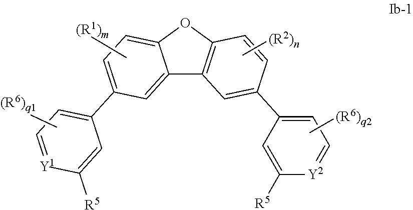
or pharmaceutically acceptable salt thereof.

8. The method of claim 7 wherein the compound of Formula Ib-1 or pharmaceutically acceptable salt thereof is a compound of Formula Ib-1-1: 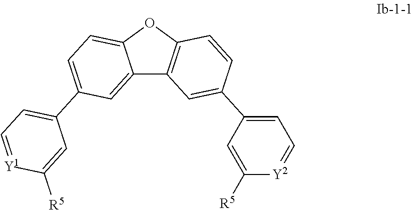
or pharmaceutically acceptable salt thereof.

9. The method of claim 6 wherein the compound of Formula Ib or pharmaceutically acceptable salt thereof is a compound that is 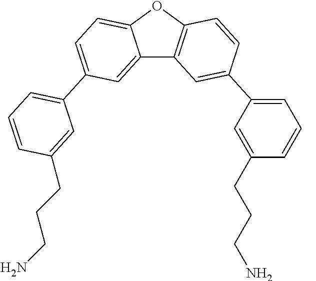
or pharmaceutically acceptable salt thereof.

10. The method of claim 1 wherein the compound of Formula II or pharmaceutically acceptable salt thereof is a compound that is: 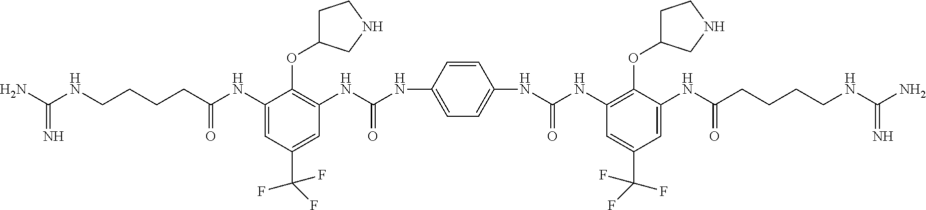
or pharmaceutically acceptable salt thereof.

11. The method of claim 1 wherein the compound of Formula III or pharmaceutically acceptable salt thereof is a compound that is: 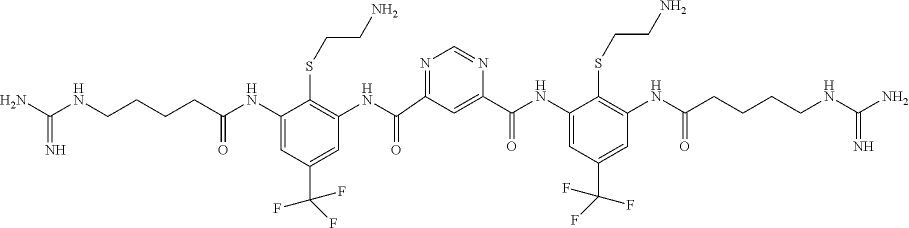
or pharmaceutically acceptable salt thereof.

12. The method of claim 1 wherein the compound of Formula IV or IVa, or pharmaceutically acceptable salt thereof, is a compound that is: 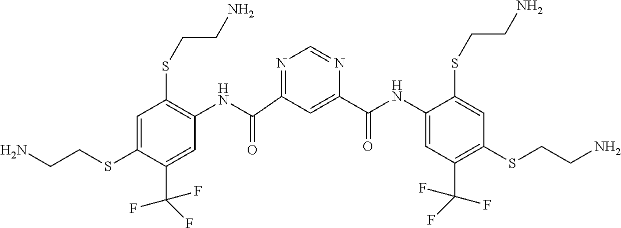
or pharmaceutically acceptable salt thereof.

13. The method of claim 1 wherein the cancer is selected from leukemia, melanoma, lung cancer, colon cancer, brain cancer, ovary cancer, breast cancer, prostate cancer, and kidney cancer.
